(19) United States
(12) Patent Application Publication
(10) Pub. No.: US 2024/0207165 A1
(43) Pub. Date: Jun. 27, 2024

(54) CONSUMER AND INDUSTRIAL PRODUCTS COMPRISING A SURFACTANT AND FATTY ACID REACTION PRODUCTS OF DEXTRINS OR DEXTRAN

(71) Applicant: Integrity Bio-Chemicals, LLC, Cresson, TX (US)

(72) Inventors: Christopher P. GARDNER, Cresson, TX (US); Stephen William ALMOND, Creston, CA (US)

(73) Assignee: Integrity Bio-Chemicals, LLC, Cresson, TX (US)

(21) Appl. No.: 18/556,821

(22) PCT Filed: Apr. 28, 2022
(86) PCT No.: PCT/US2022/026664

§ 371 Date: Oct. 23, 2023

Related U.S. Application Data
(60) Provisional application No. 63/272,368, filed on Oct. 27, 2021.
(60) Provisional application No. 63/181,523, filed on Apr. 29, 2021.

(51) Int. Cl. A61K 8/73; A61Q 19/00

(52) U.S. Cl. CPC A61K 8/732; A61Q 19/007; A61K 2800/10; A61K 2800/5922

(57) ABSTRACT

A carrier phase, a neutral surfactant or a reaction product thereof, and a reaction product of a saccharide polymer and a fatty acid or a fatty ester may be formulated as consumer or industrial products of various types. The reaction product of the saccharide polymer may be obtained in the presence of water and a hydroxide base (optionally in the presence of the neutral surfactant). The saccharide polymer comprises a dextran, a dextrin compound, or any combination thereof, and the fatty acid comprises about 50 wt. % or above of one or more straight-chain fatty acids. Illustrative products may include adjuvants, foaming agents, hard surface cleaners, skin creams and lotions, body washes, shampoos, liquid soaps, sunscreens, hair sprays and gels, cosmetics, deodorants, and antiperspirants.

### BACKGROUND

Amphiphilic compounds having both hydrophobic and hydrophilic regions within their molecular structure are commonly referred to as “surfactants” or “surfactant compounds.” By virtue of their molecular structure, surfactants tend to lower the surface tension at an interface between two components. Surfactants may be found in a wide range of consumer and industrial products including, for example, soaps, detergents, cosmetics, pharmaceuticals, and dispersants. Among other functions in these applications and others, surfactants may promote solubility of an otherwise sparingly soluble solid, facilitate surface wettability, improve dispersion of a solid, increase foaming, facilitate emulsification or de-emulsification, and/or lower viscosity in particular instances.

Some common synthetic surfactants may be expensive and subject to environmental or government regulations concerning their use, which may hamper their incorporation in products of various types that might otherwise benefit from surfactant incorporation. In addition, some surfactants may exhibit high surface tension or intrafacial tension values at the critical micelle concentration, which may complicate formulation thereof in consumer and industrial products of various types.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIGS. 1A-1D show plots of percent emulsification as a function of time for Terero oil emulsified with Samples A-D, respectively.

FIGS. 2A-2D show plots of percent emulsification as a function of time for Wolfcamp A oil emulsified with Samples A-D, respectively.

FIGS. 3A-3D show plots of surface tension as a function of concentration for Samples A-D, respectively.

FIGS. 4A-4D show plots of percent emulsification as a function of time for East Texas Hutcheson #2 oil emulsified with Samples E1-E4, F1-F4, G1-G4, and H1-H4.

FIG. 5 shows a plot of percent water emulsification at 60 minutes for Samples E1-E4, F1-F4, G1-G4, and H1-H4.

FIG. 6 shows a bar graph of Hart-DeGeorge Foam Test performance of an experimental soap formulation and a comparative soap formulation. The experimental soap formulation contains a reaction product of maltodextrin and lauric acid formed in the presence of cocamide diethanolamine, and the comparative soap formulation contains an equivalent mass of sodium lauryl sulfate, an anionic surfactant commonly used in soaps and personal care products.

### DETAILED DESCRIPTION

The present disclosure generally relates to consumer and industrial products containing surfactants and, more specifically, compositions comprising surfactants that have a low surface tension when formulated into consumer and industrial products. Consumer and industrial products may include various forms such as solutions, dispersions, creams, emulsions, foams, gels, powders, sticks, and the like.

Surfactants of various types may be found in a wide array of industrial and consumer products. However, some surfactants may be expensive, have incompatibilities with other materials, and/or be subject to regulatory constraints. High surface tension (intrafacial tension) values may be an issue for some types of surfactants as well. Moreover, there is no easy way to alter the hydrophobic-lipophilic balance (HLB) of conventional surfactants without undertaking complex chemical syntheses.

Biopolymer-based compounds may be produced through reaction of a saccharide polymer, such as a dextran and/or a dextrin compound, with a fatty acid or a fatty ester to afford reaction products that may exhibit decreased surface tension when combined with a suitable neutral surfactant. Namely, saccharide polymers comprising a dextran and/or a dextrin compound that are reacted with fatty acids or a fatty ester (e.g., a glycerol ester or other type of fatty acid precursor), preferably under alkaline conditions and optionally in the presence of the neutral surfactant, may afford reaction products having surfactant-modifying properties and unexpectedly low surface tension values when present in combination with a suitable neutral surfactant. The reaction products may be advantageous due to their biological origin, which may make the reaction products desirable for incorporation in consumer and industrial products of various types. Other advantages may include the low cost for the components used in forming the reaction products and the potential to prepare formulations exhibiting significant biodegradability. Without being limited by theory, the reaction products may include at least one fatty ester of the dextran or dextrin compound (i.e., a fatty ester saccharide polymer reaction product), which interacts synergistically with the neutral surfactant to afford the low surface tension values. Components forming the reaction products individually tend to raise surface tension values, but once all combined together in a reaction product may surprisingly lower the surface tension of cocamide diethanolamine (CocoDEA) and similar neutral surfactants, possibly after further reaction of a primary alcohol functionality of the neutral surfactant occurs. Similar neutral surfactants that may function in a like manner to CocoDEA and other cocamide-based surfactants in combination with the reaction products described herein may include, but are not limited to, other fatty acid alkanolamides, such as those formed from palmitic acid and ethanolamine or diethanolamine, for example.

Dextrin compounds have primary alcohol functionalities, as well as secondary alcohol functionalities, that may undergo a reaction with a fatty acid (optionally obtained from a fatty acid precursor, such as a fatty ester) under suitable conditions to form a reaction product, preferably a fatty ester saccharide polymer reaction product. Dextrans have secondary alcohols that may undergo a reaction to form a reaction product of similar type. The chain length of the fatty acid within the reaction products and the amount of fatty acid present when undergoing a reaction with the saccharide polymer may aid in tailoring the properties obtained therefrom in a given circumstance, such as through alteration of the hydrophobic-lipophilic balance (HLB). Reaction products having a sufficiently high HLB may promote foaming in some circumstances, for example. In other instances, a high or low HLB may promote emulsifying or non-emulsifying behavior. The ability to readily adjust the HLB of the reaction products represents a significant advantage when formulating consumer and industrial products according to the disclosure herein. Reaction products of maltodextrin represent a class of dextrin-based reaction products.

The combination of a neutral surfactant and reaction products may promote ready foaming of an aqueous fluid, and may afford a more stable foam than does a comparable mass of ionic surfactant alone, including cationic, anionic, or zwitterionic surfactants. A zwitterionic surfactant may optionally be combined with the reaction products to improve foaming performance relative to the reaction products and a neutral surfactant alone. For example, when combined with CocoDEA, other fatty acid alkanolamides or a reaction product thereof, a reaction product formed from maltodextrin and lauric acid may generate a less dense and more stable foam than does a substantially equivalent amount of sodium lauryl sulfate (sodium dodecyl sulfate), an anionic surfactant that is commonly used in personal care products such as soaps and shampoos. Given the biomolecule nature of the reaction products, foamed or foamable formulations comprising one or more reaction products of the present disclosure may offer the potential for formulating environmentally friendly soaps and other personal care products. Beyond foaming performance, zwitterionic surfactants may provide other benefits when combined with the reaction products described herein.

In addition to affording foamed or foamable formulations based upon neutral surfactant technology, the reaction products of the present disclosure may fully or partially replace more costly surfactants and/or surfactants subject to government regulations in various industrial or consumer products. For example, the reaction products of the present disclosure may be an effective replacement for ethoxylated alcohol neutral surfactants, which may contain trace levels of 1,4-dioxane. The lowering of surface tension afforded by the reaction products in combination with a neutral surfactant may be further advantageous when replacing a less desirable surfactant in a consumer or industrial product.

Maltodextrins represent advantageous saccharide polymers for use in the disclosure herein in terms of their low cost, environmentally benign nature, and the relative ease with which they may be chemically reacted with fatty acids having a range of chain lengths, which may be optionally obtained from a fatty ester (e.g., a glycerol ester) or other type of fatty acid precursor. Depending on the fatty acid or fatty ester reacted with a maltodextrin, as well as the amount thereof, the hydrophobic-lipophilic balance (HLB) of the reaction products may range from about 5 to about 20 or more, wherein known molecular contributions may be utilized to calculate the HLB value. Thus, depending on type, maltodextrin reaction products may be effective for forming emulsions in substantially water-based fluids or substantially oil-based (organic) fluids. In some examples, the maltodextrin (or dextran) reaction products may form an oil-in-water emulsion when combined with an oleaginous material in a suitable aqueous phase. In still other examples, the maltodextrin (or dextran) reaction products may promote dissolution of an oleaginous or non-oleaginous material in an aqueous phase rather than promoting emulsification. In addition to the property variation resulting from the fatty acid size and amount, maltodextrins are available in a range of oligomer sizes (e.g., 3-20 glucose monomers, or even up to about 25 glucose monomers), which may allow some further tailoring of the emulsifying or foaming properties to be realized. As such, maltodextrin reaction products may offer numerous advantages and a wide range of applicability for applications in which surfactants are commonly used, such as in consumer and industrial products. Dextran reaction products may offer similar advantages and features to those of maltodextrin reaction products.

Dextrin compounds suitable for use in the present disclosure may comprise 2 to about 20 glucose monomers, or even up to about 25 glucose monomers, linked together with α(1,4) glycosidic bonds. At least a portion of the glucose monomers may form a reaction product upon being contacted under suitable conditions with a fatty acid salt, such as a salt of a C₄-C₃₀ fatty acid or a C₄-C₂₀ fatty acid, preferably with a majority of the fatty acids residing in these size ranges being straight-chain fatty acids. A free fatty acid or a fatty ester may provide the fatty acid salt that undergoes a reaction with the saccharide polymer to form the reaction product. Without being limited by theory, at least a portion of the glucose monomers may react to form a fatty ester of the dextrin compound in some embodiments, optionally present in combination with unreacted fatty acid salt in an aqueous phase. When formed, a fatty ester dextrin reaction product may form at any hydroxyl group of the dextrin compound, including any combination of primary and/or secondary hydroxyl groups. Hydroxyl groups upon the neutral surfactant may undergo a reaction under similar conditions.

Dextran is a saccharide polymer characterized by predominantly α(1,6) glycosidic bonds between adjacent glucose monomers, with a limited number of glucose side chains linked to the main polymer backbone via α(1,3) glycosidic bonds. The α(1,3) glycosidic bonds may introduce crosslinks between adjacent saccharide polymer chains. Depending on the biological source, the extent of branching and the molecular weight of dextran may vary considerably, any of which may be utilized in the disclosure herein. At least a portion of the glucose monomers in dextran may form a reaction product upon being contacted under suitable conditions with a fatty acid salt, such a salt of a C₄-C₃₀ fatty acid or a C₄-C₂₀ fatty acid, preferably with a majority of the fatty acids residing in these size ranges being straight-chain fatty acids. A free fatty acid or a fatty ester may provide the fatty acid salt that undergoes a reaction with the saccharide polymer to form the reaction product. Without being limited by theory, at least a portion of the glucose monomers may react to form a fatty ester of the dextran in some embodiments, optionally present in combination with unreacted fatty acid salt in an aqueous phase. When formed, a fatty ester dextran reaction product may form at any hydroxyl group of the dextran.

In some embodiments, reaction products of the present disclosure may include a dextrin compound having 3 to about 20 glucose monomers, or even up to about 25 glucose monomers, that are covalently linked by α(1,4) glycosidic bonds. Formula 1 below shows the generic structure of a dextrin compound having only α(1,4) glycosidic bonds between adjacent glucose monomers, wherein variable ‘a’ is a positive integer ranging from 1 to about 18, thereby providing a dextrin backbone with 3 to about 20 glucose monomers. In the case of a dextrin compound containing up to 25 glucose monomers, variable ‘a’ may range from 1 up to about 23. The terminal glucose unit is shown in its closed form, but may also be present in the corresponding reducing sugar form as well.

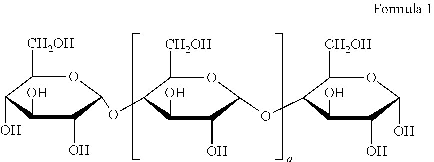

Other dextrin compounds may contain only α(1,6) glycosidic bonds or a mixture of α(1,4) and α(1,6) glycosidic bonds, and such dextrin compounds may also be suitable for use in forming the reaction products. Particularly suitable dextrins may have a molecular weight (e.g., Mn) in the range of about 1200 to about 1400 or about 1100 to about 1500.

In some or other embodiments, the reaction products may include a dextran obtained from any suitable source. The structure of dextran is shown in Formula 2 below, in which the α(1,3) glycosidic bonds are not shown in the interest of clarity. Where they occur, the α(1,3) glycosidic bonds may append a terminal glucose monomer as a side chain to the α(1,6)-linked saccharide polymer backbone, form crosslinks between adjacent α(1,6)-linked saccharide polymer backbones, interrupt the α(1,6)-linked saccharide polymer backbone with an α(1,3) glycosidic bond, or any combination thereof. Depending on source, up to about 5% of the glucose monomers may be linked by α(1,3) glycosidic bonds. Linkage by α(1,3) glycosidic bonds may occur upon any of the glucose monomers. The numbering of a single glucose monomer is shown in Formula 3 below.

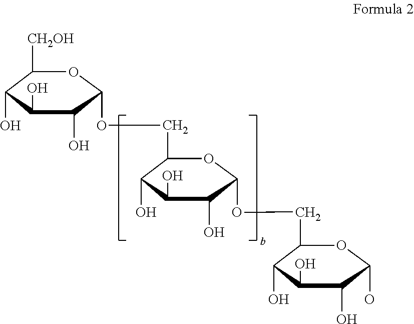

Suitable dextrans may have a molecular weight of about 1200, or about 1400, or about 5000 up to about 50,000,000 or about 100,000 up to about 20,000,000. As such, variable ‘b’ may range from about 30 to about 300,000 depending on the particular dextran selected. Particularly suitable dextrans may have a molecular weight (e.g., Mn) ranging from about 1200 to about 1400, or about 1100 to about 1500, or about 100,000 to about 1 million, or about 2 million to about 5 million. Another particularly suitable dextran may have a molecular weight of about 500,000 and an activity level of about 9%.

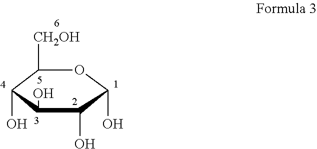

The saccharide polymer may comprise a maltodextrin according to some embodiments of the present disclosure. Maltodextrins may be characterized in terms of their dextrose equivalent (DE) value. Dextrose equivalent is a measure of the amount of reducing sugars (e.g., glucose monomers) that are present in a saccharide polymer, particularly a dextrin, expressed as a percentage relative to dextrose. Starch, which is functionally non-reducing, has a defined dextrose equivalent of 0, whereas dextrose itself has a dextrose equivalent of 100. Dextrose equivalent may be calculated by dividing the molecular weight of glucose by Mn and multiplying the result by 100. Higher dextrose equivalent values are characteristic of a lower number of covalently linked glucose monomers (shorter polymer backbone length, thereby providing a higher relative percentage of terminal reducing sugars). Maltodextrins suitable for forming a reaction product with one or more fatty acids or fatty esters according to the disclosure herein may exhibit dextrose equivalent values ranging from 3 to about 25 or from 3 to about 20. In more specific embodiments, dextrose equivalent values of the maltodextrins may range from about 4.5 to about 7.0, or from about 7.0 to about 10.0, or from about 9.0 to about 12.0.

Maltodextrins suitable for forming a reaction product may be obtained from hydrolysis or pyrolysis of starch, specifically the amylose component of starch, according to some embodiments. A maltodextrin having Formula 1 may be formed by hydrolysis or pyrolysis of amylose, for example. Alternative suitable dextrins may be obtained from hydrolysis or pyrolysis of the amylopectin component of starch, in which case the dextrin may contain α(1,6) glycosidic bonds if the dextrin is obtained through hydrolysis of the amylopectin side chain. Starches from which the dextrins may be subsequently produced may be obtained from any starch source.

Accordingly, reaction products suitable for incorporation in consumer or industrial products may comprise a first reaction component comprising a saccharide polymer selected from a dextran, a dextrin compound, or any combination thereof and a second reaction component comprising one or more fatty acids, which may be optionally obtained from a fatty acid precursor, such as a fatty ester. A majority of the one or more fatty acids may comprise one or more straight-chain fatty acids. The reaction products may be obtained in the presence of water and a hydroxide base. Suitable hydroxide bases may include, for example, alkali metal hydroxides, such as sodium hydroxide, potassium hydroxide, or any combination thereof. A stoichiometric excess or a stoichiometric deficit of the hydroxide base relative to the one or more fatty acids may be present. As such, the reaction product may further comprise a fatty acid carboxylate, such as an alkali metal carboxylate. When the reaction products are formed from a fatty ester, the reaction product may further comprise an alcohol component obtained from the fatty ester, such as glycerol from a glycerol ester. Optionally, the reaction product may be formed in the presence of a neutral surfactant, preferably where the reaction product of the saccharide polymer is present at a concentration effective to lower a surface tension of the neutral surfactant in an aqueous fluid.

Alternately, other saccharide polymers may be utilized to form the reaction products in the consumer or industrial products described herein. Other saccharide polymers that may be used in this regard include, but are not limited to, glycogen, guar, xanthan, welan, scleroglucan, chitosan, schizophyllan, levan, pectins, inulin, arabinoxylans, pullulan, gellan, carrageenan, chitosan, chitin, cellulose, starch, or a combination thereof. Saccharide polymer fragments obtained from the foregoing and containing about 3 to about 25 saccharide monomers per fragment may also be utilized for forming the reaction products described herein.

A molar ratio of fatty acid to glucose monomers in the reaction product may be about 0.05 or above on a basis of moles_{fatty acid}:moles_{glucose monomers}, or about 0.08 or above on a basis of moles_{fatty acid}:moles_{glucose monomers}, or about 0.1 or above on a basis of moles_{fatty acid}:moles_{glucose monomers}, or about 0.2 or above on a basis of moles_{fatty acid}:moles_{glucose monomers}, or about 0.3 or above on a basis of moles_{fatty acid}:moles_{glucose monomers}, or about 0.4 or above on a basis of moles_{fatty acid}:moles_{glucose monomers}, or about 0.5 or above on a basis of moles_{fatty acid}:moles_{glucose monomers}, or about 0.6 or above on a basis of moles_{fatty acid}:moles_{glucose monomers}, or about 0.7 or above on a basis of moles_{fatty acid}:moles_{glucose monomers}, or about 0.8 or above on a basis of moles_{fatty acid}:moles_{glucose monomers}, or about 0.9 or above on a basis of moles_{fatty acid}:moles_{glucose monomers}. A maximum ratio of fatty acid to dextrin or dextran in the reaction product, based upon glucose monomers, may be about 1.0 in most cases. Thus, in some embodiments, the molar ratio of fatty acid to glucose monomers in the reaction product may range from about 0.05 moles_{fatty acid}:moles_{glucose monomers} to about 1.0 moles_{fatty acid}:moles_{glucose monomers}, or about 0.05 moles_{fatty acid}:moles_{glucose monomers} to about 0.9 moles_{fatty acid}:moles_{glucose monomers}, Or about 0.05 moles_{fatty acid}:moles_{glucose monomers} to about 0.8 moles_{fatty acid}:moles_{glucose monomers}, or about 0.05 moles_{fatty acid}:moles_{glucose monomers} to about 0.7 moles_{fatty acid}:moles_{glucose monomers}, or about 0.05 moles_{fatty acid}:moles_{glucose monomers} to about 0.6 moles_{fatty acid}:moles_{glucose monomers}, or about 0.05 moles_{fatty acid}:moles_{glucose monomers} to about 0.5 moles_{fatty acid}:moles_{glucose monomers}, or about 0.05 moles_{fatty acid}:moles_{glucose monomers} to about 0.4 moles_{fatty acid}:moles_{glucose monomers}, or about 0.1 moles_{fatty acid}:moles_{glucose monomers} to about 0.9 moles_{fatty acid}:moles_{glucose monomers}, or about 0.1 moles_{fatty acid}:moles_{glucose monomers} to about 0.8 moles_{fatty acid}:moles_{glucose monomers}, Or about 0.1 moles_{fatty acid}:moles_{glucose monomers} to about 0.7 moles_{fatty acid}:moles_{glucose monomers}, or about 0.1 moles_{fatty acid}:moles_{glucose monomers} to about 0.6 moles_{fatty acid}:moles_{glucose monomers}, or about 0.1 moles_{fatty acid}:moles_{glucose monomers} to about 0.5 moles_{fatty acid}:moles_{glucose monomers}, or about 0.1 moles_{fatty acid}:moles_{glucose monomers} to about 0.4 moles_{fatty acid}:moles_{glucose monomers}, or about 0.2 moles_{fatty acid}:moles_{glucose monomers} to about 0.9 moles_{fatty acid}:moles_{glucose monomers}, or about 0.2 moles_{fatty acid}:moles_{glucose monomers} to about 0.8 moles_{fatty acid}:moles_{glucose monomers}, or about 0.2 moles_{fatty acid}:moles_{glucose monomers} to about 0.7 moles_{fatty acid}:moles_{glucose monomers}, Or about 0.2 moles_{fatty acid}:moles_{glucose monomers} to about 0.6 moles_{fatty acid}:moles_{glucose monomers}, or about 0.2 moles_{fatty acid}:moles_{glucose monomers} to about 0.5 moles_{fatty acid}:moles_{glucose monomers}, or about 0.2 moles_{fatty acid}:moles_{glucose monomers} to about 0.4 moles_{fatty acid}:moles_{glucose monomers}, or about 0.3 moles_{fatty acid}:moles_{glucose monomers} to about 0.9 moles_{fatty acid}:moles_{glucose monomers}, or about 0.3 moles_{fatty acid}:moles_{glucose monomers} to about 0.8 moles_{fatty acid}:moles_{glucose monomers}, or about 0.3 moles_{fatty acid}:moles_{glucose monomers} to about 0.7 moles_{fatty acid}:moles_{glucose monomers}, or about 0.3 moles_{fatty acid}:moles_{glucose monomers} to about 0.6 moles_{fatty acid}:moles_{glucose monomers}, Or about 0.3 moles_{fatty acid}:moles_{glucose monomers} to about 0.5 moles_{fatty acid}:moles_{glucose monomers}, or about 0.3 moles_{fatty acid}:moles_{glucose monomers} to about 0.4 moles_{fatty acid}:moles_{glucose monomers}, or about 0.4 moles_{fatty acid}:moles_{glucose monomers} to about 0.9 moles_{fatty acid}:moles_{glucose monomers}, or about 0.4 moles_{fatty acid}:moles_{glucose monomers} to about 0.8 moles_{fatty acid}:moles_{glucose monomers}, or about 0.4 moles_{fatty acid}:moles_{glucose monomers} to about 0.7 moles_{fatty acid}:moles_{glucose monomers}, or about 0.4 moles_{fatty acid}:moles_{glucose monomers} to about 0.6 moles_{fatty acid}:moles_{glucose monomers}, or about 0.4 moles_{fatty acid}:moles_{glucose monomers} to about 0.5 moles_{fatty acid}:moles_{glucose monomers}. The foregoing ratios may represent a molar ratio of fatty acid reacted with the dextran or dextrin compound. One or more hydroxyl groups per glucose monomer may undergo a reaction in some cases. At least a portion of the glucose monomers may remain unfunctionalized. Unreacted carboxylic acids, if any, may remain in the reaction product as a free carboxylate salt of the hydroxide base. As such, reaction products of the present disclosure may comprise one or more dextrin fatty esters and/or one or more dextran fatty esters, optionally in further combination with a fatty acid carboxylate (e.g., an alkali metal carboxylate), and a hydroxide base (e.g., an alkali metal hydroxide base). The hydroxide base may be present in at least a sufficient molar quantity to react with substantially all of the fatty acid that is present to form an alkali metal carboxylate. The hydroxide base may be neutralized with an acid or removed through washing, and the reaction products may maintain their ability to afford a low surface tension.

Compositions suitable for formulating a consumer or industrial product may comprise a neutral surfactant and/or a zwitterionic surfactant in combination with the foregoing reaction products. Surprisingly, the reaction products may promote lowering of the surface tension of the neutral surfactant or zwitterionic surfactant. That is, the reaction product may be present in an effective concentration to lower the surface tension relative to that produced by the neutral surfactant or the zwitterionic surfactant alone at a substantially similar concentration. Neutral surfactants may be useful due to their already-low surface tension values. When combined with the saccharide polymer during formation of the reaction product, alcohol groups upon a neutral surfactant may form a reaction product, such as with the fatty acid, as well. The compositions containing a neutral surfactant or a reaction product thereof, and the reaction product of the saccharide polymer and the fatty acid may be formulated with a carrier phase to afford consumer and industrial products of various types, including personal care products, as described in further detail below. In some embodiments, a neutral surfactant and a zwitterionic surfactant may be present in combination with one another in the consumer or industrial products described herein. The combination of a suitable neutral surfactant and a zwitterionic surfactant may enhance foamability and/or unexpectedly alter the emulsifying or de-emulsifying behavior or the reaction products.

Suitable neutral surfactants that may have their surface tension lowered in combination with a reaction product described herein include cocoamide-based surfactants such as cocamide diethanolamine, cocamide monoethanolamine, cocamide monoisopropanolamine, cocamide diisopropanolamine, and the like. Cocamide diethanolamine (CocoDEA) may be a suitable neutral surfactant for use in the disclosure herein. Other neutral surfactants that may be suitable include additional fatty acid alkanolamides, such as palmitic acid diethanolamine or monoethanolamine. Such neutral surfactants may be present at a concentration of about 20 wt. % or less, or about 10 wt. % or less, or about 5 wt. % or less, such as about 1 wt. % to about 10 wt. %, or about 3 wt. % to about 8 wt. % with respect to the entire product.

Betaine surfactants are a type of zwitterionic surfactant. Since the net charge of zwitterionic surfactants is zero, they also may be considered to constitute neutral surfactants in the disclosure herein. Zwitterionic surfactants, such as cocamidopropyl betaine, may also be present in the compositions of the present disclosure in some instances, either alone or in combination with an alkanolamide neutral surfactant, particularly when producing foamable formulations comprising the reaction products. Another type of suitable zwitterionic surfactant that may be present in combination with an alkanolamide neutral surfactant include various sultaines, such as cocoamidopropyl hydroxysultaine. Zwitterionic surfactants may likewise have their surface tension lowered when combined with the reaction products.

Once formed, the pH of the reaction products and compositions formed therefrom may reside within a range of about 1 to about 14, such as a range of about 1 to about 5, or about 5 to about 7, or about 7 to about 9, or about 9 to about 14. Lower surface tension values may be realized as the pH decreases in some instances. Decreased surface tension may also be realized in the presence of dissolved salt, such as potassium chloride.

Reaction products of a saccharide polymer, which may include those formed through a reaction of one or more fatty acids (optionally obtained from a fatty ester) with dextrin compounds and/or a dextran, may be prepared by a process comprising: heating a saccharide polymer comprising a dextran, a dextrin compound (e.g., comprising 3 to about 20 glucose monomers, or even up to about 25 glucose monomers, linked together with α(1,4) glycosidic bonds), or any combination thereof, a fatty acid (or a fatty ester) and a hydroxide base in water, obtaining a reaction product of the saccharide polymer and the fatty acid (or fatty acid obtained from a fatty ester) in an aqueous phase, and combining a neutral surfactant or optionally a reaction product thereof, such as a cocamide-based surfactant, or a zwitterionic surfactant with the reaction product in the aqueous phase. The reaction product may be combined with the neutral surfactant and/or zwitterionic surfactant in an amount effective to decrease the surface tension relative to the surfactant(s) alone at a like concentration. Any of the reaction products of a dextran or dextrin compounds may constitute a suitable saccharide polymer for forming compositions having a low surface tension. Heating may be conducted at a temperature of about 100° C. or less, such as at about 50° ° C. to about 80ºC, or about 60° ° C. to about 70° C., or about 50° ° C. to about 60° ° C.

The reaction product may be formed in the presence of the neutral surfactant and/or zwitterionic surfactant, and/or the neutral surfactant and/or zwitterionic surfactant may be combined after formation of the reaction product has been completed. For example, the reaction product may be precipitated and subsequently be redissolved in an aqueous solution containing the neutral surfactant and/or zwitterionic surfactant. In some embodiments, the reaction products may be formed in the presence of or be combined with a neutral surfactant due to the low surface tension values that may be obtained. When present during formation of the reaction product, a reaction product of a neutral surfactant having hydroxyl groups may be formed.

When a neutral surfactant is used, surface tension values for the combination of the neutral surfactant and the reaction product may be about 40 dynes/cm or less, or about 38 dynes/cm or less, or about 36 dynes/cm or less, or about 34 dynes/cm or less, or about 32 dynes/cm or less, or about 30 dynes/cm or less, or about 28 dynes/cm or less. The surface tension may be largely governed by the amount of neutral surfactant that is present (higher concentrations of neutral surfactant may afford lower surface tension values), with the chosen amount of neutral surfactant being selected to provide a desired extent of surfactancy applicable to a given application. At the chosen amount of neutral surfactant, the reaction product may be present in an amount sufficient to lower the surface tension in an aqueous phase in comparison to the surface tension that would otherwise be obtained for the surfactant alone at a substantially identical concentration in the aqueous phase. In a particular example, the surface tension may be lowered in an amount of about 10% to about 25%, or about 10% to about 20%, or about 15% to about 25%, as measured relative to the surface tension of the neutral surfactant in the aqueous phase alone at a substantially identical concentration to that present in a reaction product.

In forming the reaction products of a saccharide polymer, methods of the present disclosure may comprise combining the fatty acid (or fatty ester), the hydroxide base, and the neutral surfactant and/or zwitterionic surfactant in water to form a mixture, and heating the mixture until the fatty acid (or fatty ester) dissolves and a homogeneous mixture forms. Thereafter, the methods may comprise combining the saccharide polymer with the homogeneous mixture and continuing to heat until the reaction product has formed to a sufficient degree. The resulting aqueous mixture may be utilized directly in further applications for forming a consumer or industrial product, optionally after concentration or dilution, by being further combined with additional components targeted for a particular formulation. Formulations and products in which aqueous mixtures of the reaction products may be utilized are discussed hereinbelow. In some instances, the aqueous mixture may at least partially replace another surfactant in a specific formulation, such as a charged surfactant. In other instances, the aqueous mixture may at least partially replace an ethoxylated alcohol surfactant in a formulation.

Fatty acids suitable for use in forming reaction products of the present disclosure may be selected to afford reaction products having a range of HLB values, such as HLB values of about 5 to about 20. The fatty acids may originate from any source and range in size from about C₄ to about C₃₀, or about C₄ to about C₂₀, or about C₆ to about C₁₈, or about C₈ to about C₂₄. Suitable fatty acids for forming a reaction product according to the disclosure herein may be straight chain or branched, and saturated or unsaturated. At least a majority of the fatty acids that are reacted with the saccharide polymer may be straight-chain fatty acids (i.e., free fatty acids or fatty acids obtained from a fatty ester comprise about 50 wt. % or above of one or more straight-chain fatty acids). Preferably, the fatty acid or fatty acids obtained from a fatty ester comprise at least about 60 wt. % of one or more straight-chain fatty acids, or at least about 70 wt. % of one or more straight-chain fatty acids, or at least about 80 wt. % of one or more straight-chain fatty acids, or at least about 90 wt. % of one or more straight-chain fatty acids, or at least about 95 wt. % of one or more straight-chain fatty acids, or at least about 98 wt. % of one or more straight-chain fatty acids, or at least about 99 wt. % of one or more straight-chain fatty acids. In some embodiments, the fatty acids or fatty acids obtained from a fatty ester that are reacted with the saccharide polymer may substantially exclude branched or cyclic carboxylic acids, such that the fatty acids may consist of or consist essentially of one or more straight-chain fatty acids, such as one or more straight-chain fatty acids containing about 4 to about 30 carbon atoms. Illustrative straight-chain fatty acids that may be suitable for forming a reaction product of the present disclosure include, for example, butyric acid, valeric acid, caproic acid, enanthic acid, caprylic acid, pelabonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, trioscylic acid, lignoceric acid, pentacosylic acid, cerotic acid, carboceric acid, montanic acid, nonacosylic acid, melissic acid, crotonic acid, cervonic acid, linoleic acid, linolelaidic acid, linolenic acid, arachidonic acid, docosatetraenoic acid, myristoleic acid, palmitoleic acid, sappenic acid, vaccenic acid, paullinic acid, oleic acid, pinolenic acid, stearidonic acid, eleostearic acid, elaidic acid, gondoic acid, gadoleic acid, erucic acid, eicosenoic acid, eicosadiencoic acid, eicosatrienoic acid, eicosatetraenoic acid, docosadienoic acid, nervonic acid, mead acid, adrenic acid, the like, and any combination thereof. Lauric acid or a blend of lauric acid and myristic acid may be particularly suitable. Any branched variant of the foregoing fatty acids may also be suitably used to form a reaction product of the present disclosure, provided that the branched fatty acids are present as a minority component when forming the reaction products.

Optionally, fatty acids used to form the reaction products may be obtained from a fatty acid precursor, such as a fatty ester. As used herein, the term “fatty ester” refers to a compound containing one or more ester moieties, which comprises an alcohol component and a fatty acid component. The alcohol component may be a monohydric alcohol or a polyhydric alcohol, such as a diol or triol (e.g., glycerol). The fatty acid may be a straight chain or branched, saturated or unsaturated fatty acid, examples of which are provided hereinbelow. Preferably, suitable fatty esters may contain one or more fatty acids consisting of straight-chain fatty acids having about 4 to about 30 carbon atoms, such as the straight-chain fatty acids listed above.

To form a reaction product with the saccharide polymer, a fatty ester may undergo initial hydrolysis under alkaline conditions to generate the fatty acid component or a salt form thereof, which may then react with the saccharide polymer to form the a reaction product described herein. Alternately, the fatty ester may undergo direct transesterification with the saccharide polymer to form the a reaction product described herein. Any one or more than one of the primary or secondary alcohol functionalities upon the glucose monomer units of the saccharide polymer may undergo a reaction to form a reaction product in the disclosure herein. In the course of forming a reaction product from the saccharide polymer, the alcohol component of the fatty ester may be released into the aqueous phase in which the saccharide polymer reaction product is being formed. The alcohol component may remain present with the saccharide polymer reaction product in the aqueous phase or undergo at least partial removal therefrom. Low surface tension values may continue to be realized when an alcohol component is present in the aqueous phase in combination with the reaction product and the neutral surfactant. The alcohol component (e.g., glycerol) released into the aqueous phase may aid in solubilizing other components of the composition and/or other components blended with the compositions to make various consumer and industrial products. Additional alcohol other than that originating from a fatty ester may be present in combination with the reaction products as well.

A number of fats, oils and similar glycerol esters may serve as convenient and inexpensive sources a fatty ester used in forming reaction products described herein. Moreover, fats, oils and similar glycerol esters and amounts thereof may be selected to promote tailoring of the surfactant properties, such as altering HLB values by virtue of the fatty acid components contained therein and/or determining whether emulsifying or de-emulsifying performance results in a particular circumstance, for instance.

Suitable fatty esters for forming reaction products are not believed to be particularly limited, provided that the fatty esters undergo effective hydrolysis to release an alcohol component and one or more fatty acid components of the fatty ester upon exposure to reaction conditions used in forming the reaction products. Fatty acids originating from the fatty esters and suitable for forming reaction products of the present disclosure may be selected (through selection of a suitable fatty ester containing one or more desired fatty acids) to afford reaction products having a range of HLB values, such as HLB values of about 5 to about 20. Illustrative fatty esters are provided below. The fatty acids originating from the fatty esters may range in size from about C₄ to about C₃₀, or about C₄ to about C₂₀, or about C₆ to about C₁₈, or about C₈ to about C₂₄. Suitable fatty acids for forming a reaction product according to the disclosure herein may be straight chain or branched, and saturated or unsaturated, provided that straight-chain fatty acids comprise a majority of the fatty acids that are present in the reaction product. Illustrative fatty acids obtainable from fatty esters that may be suitable for forming a reaction product of the present disclosure include, for example, butyric acid, valeric acid, caproic acid, enanthic acid, caprylic acid, pelabonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, trioscylic acid, lignoceric acid, pentacosylic acid, cerotic acid, carboceric acid, montanic acid, nonacosylic acid, melissic acid, crotonic acid, cervonic acid, linoleic acid, linolelaidic acid, linolenic acid, arachidonic acid, docosatetraenoic acid, myristoleic acid, palmitoleic acid, sappenic acid, vaccenic acid, paullinic acid, oleic acid, pinolenic acid, stearidonic acid, eleostearic acid, elaidic acid, gondoic acid, gadoleic acid, erucic acid, eicosenoic acid, eicosadiencoic acid, eicosatrienoic acid, eicosatetraenoic acid, docosadienoic acid, nervonic acid, mead acid, adrenic acid, the like, and any combination thereof. Preferably, at least one unsaturated fatty acid, such as oleic, linoleic or linolenic acid, may be present in reaction products formed from fatty esters, since such fatty acids are commonly present in fatty esters sourced from plant or animal oils, as discussed further below.

In some embodiments, the fatty ester may comprise a glycerol ester. A glycerol ester may undergo alkaline hydrolysis to liberate glycerol as an alcohol component, and up to three carboxylic acid components per glycerol alcohol component may be released for undergoing reaction with a saccharide polymer according to the disclosure herein. The carboxylic acid components released from the glycerol ester may be the same or different, and at least one unsaturated fatty acid may be among the carboxylic acid components, according to some embodiments of the present disclosure. Preferably, the fatty acid components obtained from the fatty ester are straight-chain fatty acids.

Glycerol esters suitable for forming a reaction product in accordance with the disclosure herein are not believed to be particularly limited and may comprise any plant oil, animal oil, plant fat, animal fat, or any combination thereof that contains or more desired fatty acids. The glycerol ester may undergo hydrolysis or transesterification in the course of forming a reaction product with a saccharide polymer. Suitable glycerol esters may be found in plant or animal sources including, for example, soybean oil, grapeseed oil, olive oil, palm oil, rice bran oil, safflower oil, corn oil, coconut oil, sunflower seed oil, canola oil, rapeseed oil, peanut oil, cottonseed oil, hazelnut oil, tea seed oil, linseed oil, sesame oil, acai oil, almond oil, beech nut oil, brazil nut oil, cashew oil, macadamia nut oil, pecan oil, pine nut oil, pistachio oil, walnut oil, pumpkin seed oil, apricot oil, avocado oil, grapefruit oil, lemon oil, orange oil, mango oil, flax seed oil, fish oil, cocoa butter, hemp oil, castor oil, tall oil, fish oil, cattle fat, buffalo fat, sheep fat, goat fat, duck fat, pig fat, poultry fat, and any combination thereof.

Soybean oil, for example, contains a mixture of saturated and unsaturated fatty acids, predominantly palmitic acid, stearic acid, oleic acid, linoleic acid, and linolenic acid, with the monounsaturated and polyunsaturated fatty acids (oleic acid, linoleic and linolenic acids) comprising a majority of the fatty acids obtainable from the soybean oil. Palm oil contains about 50% saturated fatty acids (palmitic acid, stearic acid, and myristic acid) and 50% unsaturated fatty acids (oleic acid, linoleic acid, and linolenic acid). Coconut oil contains predominantly saturated fatty acids (caprylic acid, capric acid, lauric acid, myristic acid, palmitic acid, and stearic acid) and less than 10% unsaturated fatty acids (oleic acid and linoleic acid).

When glycerol esters are used as a direct (in situ) source of fatty acids for formation of reaction products of the present disclosure, glycerol may be present as the at least one alcohol in compositions containing the reaction products. Optionally, the glycerol may be at least partially removed from the aqueous phase of the compositions, if desired. Otherwise, the amount of glycerol present in the compositions may be dictated by the amount of glycerol ester that is present when forming the reaction product. For example, for glycerol esters containing C₈-C₂₄ fatty acids, the weight percentage of glycerol in the glycerol esters may range from about 7 wt. % to about 17 wt. %, based on total mass of the glycerol ester. Accordingly, the corresponding weight percentages of glycerol in compositions containing the reaction product, as measured relative to the fatty acid(s) originating from the glycerol upon alkaline hydrolysis, may range from about 7.5 wt. % to about 20 wt. %. Alternately, the weight percentage of glycerol in the compositions may be substantially equivalent on a mass basis, with respect to the entirety of the composition, to the weight percentage of glycerol ester present in the reaction mixture, since each glycerol ester may release one glycerol molecule upon undergoing complete hydrolysis.

Thus, the reaction products and consumer and industrial products formed therefrom may further comprise one or more alcohols originating from the fatty ester upon forming the reaction product of the saccharide polymer and the fatty ester. Optionally, further amounts of alcohol may be added to the compositions in addition to that released from the fatty ester in the course of forming a reaction product in accordance with the present disclosure. One or more alcohols may further be added to reaction products formed directly from free fatty acids as well, in which case an alcohol component may not be directly liberated in conjunction with forming the reaction products.

An aqueous phase containing the reaction products may be foamed after obtaining the reaction products therein, optionally after combining the reaction product with water and/or adding additional components. As used herein, the term “foam” refers to a stabilized dispersion of a large volume of gas in the form of bubbles of varying sizes in a relatively small volume of liquid. The term “foam quality” refers to the percentage of gas in a volume of foam and may be calculated by dividing the quantity (total foam volume—liquid volume) by the total foam volume. Inducing foam formation of the aqueous phase may take place by agitating the aqueous phase in the presence of a gas, such as through stirring or blending in the presence of the gas, bubbling gas through the aqueous phase, or any combination thereof. The neutral surfactant and/or zwitterionic surfactant may be present in combination with the reaction product while forming the foam.

Ionic surfactants are among the most commonly used type of surfactants for promoting foaming. However, ionic surfactants can lead to incompatibilities with other types of materials, such as divalent ions, and some may be subject to regulatory constraints, especially when used in large quantities. In addition, ionic surfactants may afford inconsistent foam performance at higher temperatures. As such, the reaction products described herein may be particularly beneficial for forming foamed or foamable formulations.

Gases suitable for forming a foam in the presence of the reaction products are not believed to be particularly limited. Suitable gases for forming a foam may include, but are not limited to, air, nitrogen, carbon dioxide, helium, natural gas, or any combination thereof. Aerosol propellants may also be used in some instances.

Foams formed according to the disclosure herein may have a foam quality of about 10% or above, or about 20% or above, or about 30% or above, or about 40% or above, or about 50% or above, or about 60% or above, or about 70% or above, or about 80% or above, or about 90% or above. The upper limit of the foam quality may be about 99%, or about 95%, or about 90%, or about 80%, or about 70%, or about 60% or about 50%.

Foamed or foamable formulations of the present disclosure may comprise an aqueous phase comprising an aqueous carrier fluid, which is described in more detail hereinafter. Foamed formulations (foams) are compositions to which a gas has already been introduced and foam bubbles have formed. That is, foamed formulations may comprise a gas, and an aqueous fluid comprising a composition described herein admixed together with the gas as a plurality of bubbles. Foamable formulations, in contrast, are compositions suitable for forming a foam once a gas has been introduced thereto, but which have not yet formed foam bubbles.

In addition to the reaction products, foamed or foamable formulations may further comprise one or more additional surfactants, which may be cationic, anionic, zwitterionic, neutral, or any combination thereof. Foamed or foamable formulations may also contain additional components found in soaps and other personal care products, examples of which will be familiar to persons having ordinary skill in the art. Additional disclosure directed to industrial and consumer products, including personal care products, in which the compositions described herein may be present are discussed in further detail below.

Reaction products may be provided, sourced, mixed, or stored in solid form or in liquid form when present in a consumer or industrial product. Liquid forms may be disposed in a suitable fluid phase, such as an aqueous phase, which may be emulsified or non-emulsified depending on particular formulations and intended applications. In addition, the aqueous phase may be foamed in some instances. As used herein, the terms “fluid” and “fluid phase” refer to both liquids and gels, including solutions, emulsions and suspensions of the reaction products, including foams, unless otherwise indicated. Compositions including a reaction product of the present disclosure may comprise an aqueous carrier fluid. Suitable aqueous carrier fluids may include, for example, fresh water, acidified water, seawater, brine (i.e., a saturated salt solution), or an aqueous salt solution (i.e., a non-saturated salt solution). Water-miscible organic co-solvents such as ethanol or ethylene glycol, for example, may be present in combination with an aqueous carrier fluid, in some embodiments. Suitable aqueous carrier fluids may be present while forming the reaction products, or an aqueous carrier fluid may be introduced to the reaction products following their formation.

The compositions of the present disclosure may be incorporated in various consumer and industrial products, as discussed in further detail hereinafter. Illustrative consumer and industrial products may include, but are not limited to, adjuvants, foaming agents, hard surface cleaners, skin creams, lotions, body washes, shampoos, liquid soaps, sunscreens, hair gels, hair sprays, hair dyes, cosmetics, deodorants, antiperspirants, and like personal care products. Personal care products may represent a beneficial class of products in which the compositions of the present disclosure may be present, given the relative benign nature of the biomolecules present in the compositions disclosed herein. Suitable forms of these consumer products may include liquid forms, solid forms, powder forms, gel forms, cream forms, solution forms, suspension forms, stick forms, and the like. A carrier phase suitable to produce a desired form for the consumer or industrial products may be combined with the neutral surfactant or the reaction product thereof, and the reaction product of the saccharide polymer and fatty acid. Suitable carrier phases may include aqueous liquids, organic solvents, waxes, oils, polymers, emulsions, the like and any combination thereof.

Accordingly, consumer or industrial products described herein may comprise a carrier phase, a neutral surfactant or a reaction product form thereof combined with the carrier phase, and a reaction product of the saccharide polymer and a fatty acid or fatty ester combined with the carrier phase, in which the saccharide polymer comprises a dextran, a dextrin compound, or any combination thereof, and the fatty acid comprises about 50 wt. % or above of one or more straight-chain fatty acids. Exemplary consumer and industrial products and components for such consumer and industrial products containing the reaction products are provided hereinafter.

### Adjuvants

Adjuvants are compositions that are used in combination with an active substance to increase the efficacy or potency of the active substance. In non-limiting examples, the active substance may be a pharmaceutical compound, a personal care compound, or an agricultural compound.

A reaction product of a dextrin compound or a dextran and a fatty acid, as specified above, in combination with a neutral surfactant or a zwitterionic surfactant or a reaction product thereof may be present in adjuvant compositions in which surfactants of various types may be used. The compositions of the disclosure herein may replace a surfactant used in an adjuvant composition or be used in combination with a surfactant already present in an adjuvant composition, including partial replacement of an existing surfactant. Within an adjuvant composition, compositions comprising the reaction products and a surfactant may be present in an amount of about 0.01 wt. % to about 20 wt. % of the adjuvant composition as a whole, or about 0.1 wt. % to about 10 wt. %, or about 1 wt. % to about 15 wt. %, or about 5 wt. % to about 20 wt. %.

An active compound may be present in the adjuvant compositions, or an adjuvant composition may be administered separately from an active compound. When administered separately, the adjuvant compositions may be administered before or after the active compound. Suitable active compounds may include, but are not limited, to, herbicides, pharmaceuticals, and the like, and particular examples of such active compounds are not believed to be particularly limited.

Examples of suitable additional components that may be present in adjuvant compositions containing a reaction product of the present disclosure include, but are not limited to, other surfactants, anti-foam compounds, particulates, metal oxides (e.g., silica, alumina, titania, zirconia, and the like), electrolytes, salts, organic solvents, wetting agents, dispersants, emulsifying agents, de-emulsifying agents, penetrants, preservatives, colorants, acids, bases, buffers, chelating agents, viscosifiers, thixotropic agents, stabilizers, film-forming agents, plasticizers, preservatives, antioxidants, and the like, including any combination thereof. Suitable examples of these additional components will be familiar to one having ordinary skill in the art. Other surfactants that may be present in the adjuvant compositions are not particularly limited and may include any one or a combination of cationic, anionic, neutral, or zwitterionic surfactants.

In addition, agricultural adjuvant compositions may comprise one or more of a herbicide, a molluscicide, a fungicide, a plant growth regulator, and a safener, or any combination thereof.

Some examples of adjuvant compositions comprising a reaction product of a saccharide polymer may include compositions containing another surfactant. Alternately, the neutral or zwitterionic surfactant introduced with the reaction product may constitute the entirety of the surfactants in the adjuvant compositions. Other surfactants may be present in an amount up to about 20 wt. % of the adjuvant composition.

Some examples of the adjuvant compositions may comprise an oil-in-water emulsion. Other examples of the adjuvant compositions may comprise a water-in-oil emulsion. Other suitable forms of the adjuvant compositions may comprise a solution, suspension, gel, cream, or similar formulation. Adjuvant compositions may be delivered by spraying, injection, ingestion, implantation, or other related delivery routes.

Some examples of the adjuvant compositions may include the reaction product of the saccharide polymer in combination with one or more inorganic salts, such as potassium chloride, sodium chloride, or the like. A ratio of reaction product to inorganic salts may range from about 1:99 to 99:1 on a weight basis.

### Foaming Agents

Foaming agents are compositions that are a stabilized dispersion of a large volume of gas in the form of bubbles of varying sizes in a relatively small volume of liquid, or compositions that may form a foam upon suitable introduction of gas thereto (foamable formulations).

A reaction product of a dextrin compound or a dextran and a fatty acid or a fatty ester, as specified above, in combination with a neutral surfactant or a zwitterionic surfactant, including the combination of a neutral surfactant and a zwitterionic surfactant in the case of forming a foam, may be present in foaming agents in which surfactants of various types may be used. The compositions may replace a surfactant used in a foaming agent or be used in combination with a surfactant already present in a foaming agent. Within a foaming agent, compositions comprising the reaction products and a surfactant may be present in an amount of about 0.01 wt. % to about 20 wt. % of the foaming agent as a whole, or about 0.1 wt. % to about 10 wt. %, or about 1 wt. % to about 15 wt. %, or about 5 wt. % to about 20 wt. %.

Foaming agents may contain any combination of cationic surfactants, anionic surfactants, zwitterionic surfactants, or neutral surfactants. The compositions disclosed herein may be present in a foaming agent in combination with any of cationic surfactants, anionic surfactants, zwitterionic surfactants, neutral surfactants or any two or more of these surfactants. Alternately or additionally, the compositions disclosed herein may replace all or a portion of any one or more of these surfactants in a foaming agent. For example, the compositions of the present disclosure may replace anionic surfactants used in combination with zwitterionic surfactants in a foaming agent. That is, the compositions may be present in a foaming agent in combination with one or more zwitterionic surfactants. The compositions may replace a sulfosuccinate surfactant or be used in combination with a sulfosuccinate surfactant in some foaming agent embodiments. Ethoxylated alcohol surfactants may be replaced similarly by the reaction products disclosed herein.

Examples of suitable additional components that may be present in foaming agents containing a reaction product of the present disclosure include, but are not limited to, other surfactants, amines (any one or a combination of primary amines, secondary amines, tertiary amines, diethanolamine, triethanolamine, ethoxylated amines and amidoamines), foam boosters such as amine oxides, solvents, water, salts, skin conditioners (e.g., ethylhexylglycerin, hydroxyethylurea, urea, panthenol, glycerin, isopropyl myristate, propylene glycol, tocopheryl acetate, and polyquaternium-11), moisturizers, liquefied gases, supercritical gases, acids, bases, salts, buffers, chelating agents, and the like, including any combination thereof. Suitable examples of these additional components will be familiar to one having ordinary skill in the art. Other surfactants that may be present in the foaming agents are not particularly limited and may include any one or a combination of cationic, anionic, neutral, or zwitterionic surfactants.

Some examples of foaming agents comprising a reaction product of the present disclosure may include compositions containing another surfactant. Alternately, the neutral or zwitterionic surfactant introduced with the reaction product may constitute the entirety of the surfactants in the foaming agents. Other surfactants may be present in an amount up to about 20 wt. % of the foaming agents.

Some examples of the foaming agents may comprise an oil-in-water emulsion. Other examples of the foaming agents may comprise a water-in-oil emulsion. Other suitable forms of the foaming agents may comprise a solution, suspension, gel, cream, or similar formulation.

Some examples of the foaming agents may include the reaction product of the saccharide polymer in combination with one or more inorganic salts, such as potassium chloride, sodium chloride, or the like. A ratio of reaction product to inorganic salts may range from about 1:99 to 99:1 on a weight basis.

Some examples of the foaming agents may include the reaction product of the saccharide polymer in combination with a zwitterionic surfactant, such as a betaine surfactant.

Some examples of the foaming agents may include the reaction product of the saccharide polymer in combination with one or more primary amines, secondary amines, tertiary amines, amidoamines, alkanolamines, amine oxides, or any combination thereof.

Some examples of the foaming agents may include the reaction product of the saccharide polymer in combination with an aerosol propellant. Suitable aerosol propellants may include compressed gases such as carbon dioxide, nitrogen, nitrous oxide, volatile hydrocarbons (e.g., ethane, propane, butane, or isobutane), dimethyl ether, ethyl methyl ether, hydrofluorocarbons, hydrofluoroolefins, or any combination thereof.

Personal care products such as shaving cream may contain foaming agents including reaction products of the present disclosure. When included in a shaving cream or similar personal care product, skin conditioners such as ethylhexylglycerin, hydroxyethylurea, urea, panthenol, glycerin, isopropyl myristate, propylene glycol, tocopheryl acetate, polyquaternium-11, or any combination thereof may be present.

### Hard Surface Cleaners

Hard surface cleaners are compositions that may be used to remove various substances from surfaces like glass, metals, plastics, stone, concrete, and the like. Hard surfaces that may be cleaned with hard surface cleaners include, for example, windows, countertops, appliances, floors, driveways, toilets, showers and bathtubs, sinks, and the like. Substances removable from these types of hard surfaces and others span a wide range and include, but are not limited to, dirt, grease, soap scum, limescale and similar hard water deposits, and the like.

A reaction product of a dextrin compound or a dextran and a fatty acid or a fatty ester, as specified above, in combination with a neutral surfactant or a zwitterionic surfactant may be present in hard surface cleaners in which surfactants of various types may be used. The compositions of the disclosure herein may replace a surfactant used in a hard surface cleaner or be used in combination with a surfactant already present in a hard surface cleaner. Within a hard surface cleaner, compositions comprising the reaction products and a surfactant may be present in an amount of about 0.01 wt. % to about 20 wt. % of the hard surface cleaner as a whole, or about 0.1 wt. % to about 10 wt. %, or about 1 wt. % to about 15 wt. %, or about 5 wt. % to about 20 wt. %.

Examples of suitable additional components that may be present in hard surface cleaners containing a reaction product of the present disclosure include, but are not limited to, other surfactants, foaming compounds, anti-foam compounds, salts such as alkali metal carbonates, organic solvents such as glycols or glycol ethers, wetting agents, dispersants, emulsifying agents, de-emulsifying agents, colorants, acids, bases, buffers, chelating agents, anti-streaking agents, alkanolamines, and the like, including any combination thereof. Suitable examples of these additional components will be familiar to one having ordinary skill in the art. Other surfactants that may be present in the hard surface cleaners are not particularly limited and may be any one or a combination of cationic, anionic, neutral, or zwitterionic surfactants.

Some examples of hard surface cleaners comprising a reaction product of the present disclosure may include compositions containing another surfactant. Alternately, the neutral or zwitterionic surfactant introduced with the reaction product may constitute the entirety of the surfactants in the hard surface cleaners. Other surfactants may be present in an amount up to about 20 wt. % of the hard surface cleaners.

Some examples of the hard surface cleaners may comprise an oil-in-water emulsion. Other examples of the hard surface cleaners may comprise a water-in-oil emulsion. Still other examples of hard surface cleaners may comprise an aqueous solution containing the reaction product and other components in a dissolved state. Other suitable forms of hard surface cleaners may comprise a powder, suspension, gel, cream, or similar formulation.

Some examples of the hard surface cleaners may include the reaction product of the saccharide polymer in combination with one or more inorganic salts, such as potassium chloride, sodium chloride, sodium carbonate, sodium bicarbonate, potassium carbonate, potassium bicarbonate, or the like. A ratio of reaction product to inorganic salts may range from about 1:99 to 99:1 on a weight basis.

Some examples of the hard surface cleaners may include the reaction product of the saccharide polymer in combination with one or more primary amines, secondary amines, tertiary amines, amidoamines, alkanolamines, amine oxides, or any combination thereof.

Some examples of the hard surface cleaners may be aqueous solutions having a pH ranging from about 1 to about 5, or about 5 to about 9, or about 9 to about 12, or about 12 to about 14.

Some examples of the hard surface cleaners may include the reaction product of the saccharide polymer in combination with an alcohol, glycol, or glycol ether. Up to about 25 wt. %, or up to about 10 wt. % alcohol, glycol, or glycol ether may be present. Some examples of the hard surface cleaners may include up to about 10 wt. % chelating agent or up to about 5 wt. % chelating agent in combination with the reaction product of the saccharide polymer.

### Skin Creams and Lotions

Skin creams and lotions are compositions that may moisturize or otherwise improve the appearance of skin. Skin creams and lotions are inclusive of gel formulations for application to the skin, which may have a higher viscosity than creams or lotions.

A reaction product of a dextrin compound or a dextran and a fatty acid or a fatty ester, as specified above, in combination with a neutral surfactant or a zwitterionic surfactant may be present in skin creams and lotions in which surfactants may be used. The compositions may replace a surfactant used in a skin cream or lotion or be used in combination with a surfactant already present in a skin cream or lotion. Within a skin cream or lotion, compositions comprising the reaction products and a surfactant may be present in an amount of about 0.01 wt. % to about 20 wt. % of the skin cream or lotion as a whole, or about 0.1 wt. % to about 10 wt. %, or about 1 wt. % to about 15 wt. %, or about 5 wt. % to about 20 wt. %.

Examples of suitable additional components that may be present in skin creams or lotions disclosed herein include, but are not limited to, other surfactants, emulsifers, essential oils, waxes, fats, solvents, viscosifying agents, mono-alcohols, diols, polyols, diol and polyol ethers, milk proteins, emollients, humectants, skin conditioners, preservatives, acids, bases, buffers, chelating agents, thickeners, vitamins, lubricants, wrinkle reducers, moisturizers, radical inhibitors and other antioxidants, Vitamin A, Vitamin E, ceramides, fatty acids, fatty esters, fatty alcohols, hyaluronic acid, sodium pyroglutamic acid, glycerin, aloe vera, fragrances, colorants, preservatives, sunscreens, and the like, including any combination thereof. Suitable examples of these additional components will be familiar to one having ordinary skill in the art. Other surfactants that may be present in the skin creams and lotions are not particularly limited and may be any one or a combination of cationic, anionic, neutral, or zwitterionic surfactants. The reaction products may replace at least a portion of one or more existing surfactants in a skin cream or lotion or supplement a quantity of one or more existing surfactants in a skin cream or lotion.

Some examples of skin creams or lotions comprising a reaction product of the present disclosure may include compositions containing another surfactant. Alternately, the neutral or zwitterionic surfactant introduced with the reaction product may constitute the entirety of the surfactants in the skin creams or lotions. Other surfactants may be present in an amount up to about 20 wt. % of the skin creams or lotions.

Some examples of the skin creams or lotions may comprise an oil-in-water emulsion. Other examples of the skin creams or lotions may comprise a water-in-oil emulsion. Other suitable forms of skin creams or lotions may comprise a gel, suspension, or similar formulation.

Some examples of the skin creams or lotions may include the reaction product of the saccharide polymer in combination with one or more inorganic salts, such as potassium chloride, sodium chloride, or the like. A ratio of reaction product to inorganic salts may range from about 1:99 to 99:1 on a weight basis.

Some examples of the skin creams or lotions may include the reaction product of the saccharide polymer in combination with a buffer. Some examples of the skin creams or lotions may include the reaction product of the saccharide polymer in combination with a chelating agent.

Some examples of the skin creams or lotions may include the reaction product of the saccharide polymer in combination with Vitamin E or hyaluronic acid.

Some examples of the skin creams or lotions may include the reaction product of the saccharide polymer in combination with one or more oils (including essential oils), waxes, or fats. Suitable oils may include, for example, dimethicone, squalane, a phenyltrimethicone, triethylhexanoin, and any combination thereof. Essential oils may include, for example, chamomile oil, lavender oil, tea tree oil, and similar fragrant oils. Glycerin may also be present in any of the skin creams or lotions.

Some examples of the skin creams or lotions may include the reaction product of the saccharide polymer in combination with one or more milk proteins, emollients, humectants, or skin conditioners.

Some examples of the skin creams or lotions may include the reaction product of the saccharide polymer in combination with one or more thickeners. Suitable thickeners may include, but are not limited to, synthetic polymers, acrylate copolymers, acrylates crosspolymers, acrylic acid copolymers, adipic acid copolymers, polyethylene, polyvinyl acetate, polyvinyl alcohol, polyvinyl chloride, polyacrylamide and any copolymer thereof.

### Body Washes, Shampoos and Liquid Soaps

Body washes and shampoos are cleansing compositions formulated for application to the skin or hair. Liquid soaps for more generalized personal cleansing, such as for washing one's hands, are similar in composition to some body washes and shampoos and may be formulated with many of the same components.

A reaction product of a dextrin compound or a dextran and a fatty acid or a fatty ester, as specified above, in combination with a neutral surfactant or a zwitterionic surfactant may be present in body washes, shampoos and liquid soaps in which surfactants may be used. The compositions of the disclosure herein may replace a surfactant used in a body wash, shampoo, or liquid soap or be used in combination with a surfactant already present in a body wash, shampoo or liquid soap. Within a body wash, shampoo, or liquid soap, compositions comprising the reaction products and a surfactant may be present in an amount of about 0.01 wt. % to about 20 wt. % of the adjuvant composition as a whole, or about 0.1 wt. % to about 10 wt. %, or about 1 wt. % to about 15 wt. %, or about 5 wt. % to about 20 wt. %.

Examples of suitable additional components that may be present in body washes, shampoos, or liquid soaps disclosed herein include, but are not limited to, other surfactants, conditioners, amidoamines, fragrances, colorants, essential oils, foaming agents, humectants, fatty acids, fatty esters, fatty alcohols, waxes, biocides, soaps, preservatives, acids, bases, buffers, chelating agents, thickeners, vitamins, pearlizing agents, viscosifying agents, moisturizers, antioxidants, sunscreens, and the like, including any combination thereof. Suitable examples of these additional components will be familiar to one having ordinary skill in the art. Other surfactants that may be present in the body washes, shampoos and liquid soaps are not particularly limited and may be any one or a combination of cationic, anionic, neutral, or zwitterionic surfactants. The reaction products may replace at least a portion of one or more existing surfactants in body washes, shampoos and liquid soaps or supplement a quantity of one or more existing surfactants in body washes, shampoos and liquid soaps.

Suitable fatty acids and fatty esters may include, but are not limited to, (C₂-C₄)alkoxylated mono(C₂-C₃)alkanol isostearamide, polyethoxylated glyceryl fatty acid esters, polyethoxylated esters of fatty acids and saccharides PPG-2 hydroxyethyl coco/isostearamide, PEG-18 glyceryl oleate/cocoate, PEG-7 glyceryl cocoate, PEG-120 methyl glucose dioleate, and any combination thereof.

Illustrative examples of body washes, shampoos, and liquid soaps may comprise water, an effective amount of the compositions, optionally in further combination with another surfactant, 0-4% pearlizing agent, 0-1% suspension aids, 0-2% fragrance, 0-0.25% chelating agent, 0-1% preservatives, 0-2% colorant and 0-25% conditioner. Other surfactants that may be present in the body washes, shampoos, and liquid soaps are not particularly limited and may be any one or a combination of cationic, anionic, neutral, or zwitterionic surfactants.

Some examples of body washes, shampoos and liquid soaps comprising a reaction product of the present disclosure may include compositions containing another surfactant. Alternately, the neutral or zwitterionic surfactant introduced with the reaction product may constitute the entirety of the surfactants in the body washes, shampoos and liquid soaps. Other surfactants may be present in an amount up to about 20 wt. % of the body washes, shampoos and liquid soaps.

Some examples of the body washes, shampoos and liquid soaps may comprise an oil-in-water emulsion. Other examples of the body washes, shampoos and liquid soaps may comprise a water-in-oil emulsion. Other suitable forms of body washes, shampoos and liquid soaps may comprise a powder, suspension, gel, cream, or similar formulation

Some examples of the body washes, shampoos and liquid soaps may include the reaction product of the saccharide polymer in combination with one or more inorganic salts, such as potassium chloride, sodium chloride, or the like. A ratio of reaction product to inorganic salts may range from about 1:99 to 99:1 on a weight basis.

Some examples of the body washes, shampoos and liquid soaps may include the reaction product of the saccharide polymer in combination with a buffer. Some examples of the body washes, shampoos and liquid soaps may include the reaction product of the saccharide polymer in combination with a chelating agent.

Some examples of the body washes, shampoos and liquid soaps may include the reaction product of the saccharide polymer in combination with a conditioner, moisturizer, or any combination thereof.

Some examples of the body washes, shampoos and liquid soaps may include the reaction product of the saccharide polymer in combination with a pearlizing agent, humectant or a foaming agent. Suitable humectants may include, for example, glycerin or aloe vera. About 1 wt. % to about 15 wt. % humectant may be present.

Some examples of the body washes, shampoos and liquid soaps may include the reaction product of the saccharide polymer in combination with a sunscreen.

Some examples of the body washes, shampoos and liquid soaps may include the reaction product of the saccharide polymer in combination with an amidoamine.

Some examples of the body washes, shampoos, and liquid soaps may include the reaction product of the saccharide polymer in combination with Vitamin E, aloe vera gel, tea tree extract, peppermint extract, anise extract, valerian extract, lemongrass oil, calendula extract, nettle extract, lavender extract, rose extract, lemon juice, lime juice, grapefruit juice, or any combination thereof.

Some examples of the body washes, shampoos, and liquid soaps may include the reaction product of the saccharide polymer in combination with one or more oils (including essential oils), waxes, or fats. Suitable oils may include, for example, dimethicone, squalane, a phenyltrimethicone, p triethylhexanoin, and any combination thereof. Essential oils may include, for example, chamomile oil, lavender oil, tea tree oil, and similar fragrant oils. Glycerin may also be present in any of the body washes, shampoos, and liquid soaps.

### Sunscreens

Sunscreens are substances that may be applied to the skin to afford protection from the sun. Sunscreens may be formulated as creams or with a suitable wax base in “stick” form for application to the skin.

A reaction product of a dextrin or dextran and a fatty acid or a fatty ester, as specified above, in combination with a neutral surfactant or a zwitterionic surfactant may be present in sunscreens in which surfactants may be used. The compositions of the disclosure herein may replace a surfactant used in a sunscreen or be used in combination with a surfactant already present in a sunscreen. Within a sunscreen, compositions comprising the reaction products and a surfactant may be present in an amount of about 0.01 wt. % to about 20 wt. % of the sunscreen composition as a whole, or about 0.1 wt. % to about 10 wt. %, or about 1 wt. % to about 15 wt. %, or about 5 wt. % to about 20 wt. %.

Examples of suitable additional components that may be present in sunscreens include, but are not limited to, other surfactants, conditioners, titanium dioxide, zinc oxide, organic UV absorbers, film forming agents, solvents, aerosol propellants, waxes, fats, oils, moisturizers, fragrances, colorants, essential oils, fatty acids, fatty esters, fatty alcohols, preservatives, acids, bases, buffers, chelating agents, thickeners, insect repellents, skin conditioners, and the like, including any combination thereof. Suitable examples of these additional components will be familiar to one having ordinary skill in the art. Other surfactants that may be present in the sunscreens are not particularly limited and may be any one or a combination of cationic, anionic, neutral, or zwitterionic surfactants. The reaction products may replace at least a portion of one or more existing surfactants in a sunscreen or supplement a quantity of one or more existing surfactants in a sunscreen.

Organic UV absorbers that may be present in a sunscreen in combination with the compositions include, but are not limited to, para-aminobenzoic acid, avobenzone, cinoxate, dioxybenzone, homosalate, menthyl anthranilate, octyl salicylate, oxybenzone, padimate O, phenylbenzimidazole sulfonic acid, sulisobenzone, trolamine salicylate, diethanolamine methoxycinnamate, digalloy trioleate, ethyl dihydroxypropyl PABA, glyceryl aminobenzoate, lawsone with dihydroxyacetone, red petrolatum, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, isopentyl 4-methoxycinnamate, phenylbenzimidazole sulfonate, 2-hydroxy-4-methoxy benzophenone-5-sulfonate, 4-(2-beta-glucopyrano-siloxy)propoxy-2-hydroxybenzophenone, and bis-sodium phenylene-1,4-bis(2-benzimidazyl)-3,3′-5,5′-tetrasulfonate, 2-ethylhexyl-p-methoxycinnamate, 4-tert-4′-methoxydibenzoylmethane, octocrylene, 2,4-bis-[{4-(2-ethythexyloxy)-2-hydroxy}-phenyl]-6-(4-methoxyphenyl)-1,3,5-triazine, methylene bis-benzotriazolyl tetrarnethyl butylphenol, 2,4,6-tris-[4-(2-ethylhexyloxycarbonyl)anilino]-1,3,5-triazine, diethylamino hydroxybenzoyl hexyl benzoate, oxybenzone, and dihydroxy dimethoxy benzophenone, and mixtures thereof.

Still other organic UV absorbers that may be suitable for inclusion in a sunscreen include, but are not limited to, bis-resorcinyl triazines; benzimidazole derivatives; 4-methylbenzylidene camphor; benzoyl piperazine derivatives; benzoxazole derivatives; diarylbutadiene derivatives; phenyl benzotriazole derivatives; benzylidene malonates; TEA-salicylate; imidazoline derivatives; naphthalates; merocyanine derivatives; aminobenzophenone derivatives; dibenzoylmethane derivatives; β,β-diphenylacrylate derivatives; camphor derivatives; salicylate derivatives; anthranilate derivatives; and benzalmalonate derivatives.

In addition to formulations that are sunscreens alone, the compositions of the present disclosure may be present in sunscreens that are incorporated into other products such as lotions, cologne, cosmetics, body washes and shampoos, and the like.

Some examples of sunscreens comprising a reaction product of the present disclosure may include compositions containing another surfactant. Alternately, the neutral or zwitterionic surfactant introduced with the reaction product may constitute the entirety of the surfactants in the sunscreens. Other surfactants may be present in an amount up to about 20 wt. % of the sunscreens.

Some examples of the sunscreens may comprise an oil-in-water emulsion. Other examples of sunscreens may comprise a water-in-oil emulsion. Other suitable forms of skin creams or lotions may comprise a solution, gel, suspension, powder, or similar formulation.

Some examples of the sunscreens may include the reaction product of the saccharide polymer in combination with one or more inorganic salts, such as potassium chloride, sodium chloride, or the like. A ratio of reaction product to inorganic salts may range from about 1:99 to 99:1 on a weight basis.

Some examples of the sunscreens may include the reaction product of the saccharide polymer in combination with a buffer. Some examples of the sunscreens may include the reaction product of the saccharide polymer in combination with a chelating agent.

Any embodiment of a sunscreen may include the reaction product of the saccharide polymer in combination with an inorganic UV absorbing materials (e.g., titanium dioxide) and/or an organic UV absorbing material. SPF values for the sunscreens may be about 2 or above, or about 10 or above, or about 20 or above, or about 30 or above, or about 50 or above, or about 100 or above.

Some examples of sunscreens may comprise the reaction product of the saccharide polymer, up to 30 wt. % surfactants in total, up to 95 wt. % water in total, up to about 80 wt. % oil in total, and up to about 15 wt. % UV absorber in total.

### Hair Gels and Hair Sprays

Hair gels and hair sprays are formulations that may be used for holding one's hair in place, or optionally to provide detangling of one's hair. Hair sprays are aerosolized formations, whereas hair gels are high-viscosity fluids and may be applied by hand.

A reaction product of a dextrin or dextran and a fatty acid or a fatty ester, as specified above, in combination with a neutral surfactant or a zwitterionic surfactant may be present in hair sprays and hair gels in which surfactants may be used. The compositions of the disclosure herein may replace a surfactant used in a hair spray or hair gel or be used in combination with a surfactant already present in a hair spray or hair gel. Within a hair spray or hair gel, compositions comprising the reaction products and a surfactant may be present in an amount of about 0.01 wt. % to about 20 wt. % of the hair sprays or hair gels as a whole, or about 0.1 wt. % to about 10 wt. %, or about 1 wt. % to about 15 wt. %, or about 5 wt. % to about 20 wt. %.

Examples of suitable additional components that may be present in hair sprays or hair gels include, but are not limited to, other surfactants, cellulose-based biopolymers, water-soluble polymers, polyalkylene glycols, polyalkylene glycol esters, conditioning agents, emollients, humectants, emulsifiers, opacifying agents, thickening agents, foam stabilizers, viscosity builders, sequestrates, antioxidants, antidandruff agents, suspending agents, proteins, fragrances, sunscreens, botanical extracts, essential oils, fatty acids, fatty esters, fatty alcohols, preservatives, acids, bases, buffers, chelating agents, thickeners, vitamins, waxes, oils, aerosol propellants, polyvinylpyrrolidone, polyvinyl acetate, vinyl acetate-crotonic acid copolymers, acrylic acid copolymers, plasticizers, alcohols, and the like, including any combination thereof. Examples of suitable additional components will be familiar to one having ordinary skill in the art. Other surfactants that may be present in the hair sprays and hair gels are not particularly limited and may be any one or a combination of cationic, anionic, neutral, or zwitterionic surfactants. The reaction products may replace at least a portion of one or more existing surfactants in a hair spray or hair gel or supplement a quantity of one or more existing surfactants in a hair spray or hair gel.

Some examples of hair sprays and hair gels comprising a reaction product of the present disclosure may include compositions containing another surfactant. Alternately, the neutral or zwitterionic surfactant introduced with the reaction product may constitute the entirety of the surfactants in the hair sprays and hair gels. Other surfactants may be present in an amount up to about 20 wt. % of the hair sprays and hair gels.

Some examples of the hair sprays and hair gels may comprise an oil-in-water emulsion. Other examples of the hair sprays and hair gels may comprise a water-in-oil emulsion. Still other examples of hair sprays and hair gels may comprise an aqueous solution containing the reaction product and other components in a dissolved state.

Some examples of the hair sprays and hair gels may comprise the reaction product of the saccharide polymer in combination with a polymer, such as polyvinylpyrrolidone, polyvinyl acetate, vinyl acetate-crotonic acid copolymers, and acrylic acid copolymers. Other example polymers that may be present include polyquaterniums-10, polyquaterniums-24, polyquaterniums-27, polyquaterniums-67, polyquaterniums-72, and mixtures thereof.

Some examples of hair sprays and hair gels may comprise the reaction product of the saccharide polymer in combination with an aerosol propellant. Some examples of hair sprays and hair gels may comprise the reaction product of the saccharide polymer in combination with a foaming agent.

One or more examples of a hair spray or hair gel may comprise a composition of the present disclosure and one or more of cetearyl alcohol, behentrimonium chloride, cyclopentasiloxane, dimethicone, ethylhexyl isononanoate, behenyl alcohol, meadowfoam seed oil, cyclohexasiloxane, olive fruit oil, prunus amygdalus dulcis, stearamidopropyl dimethylamine, behentrimonium methosulfate, amodimethicone, panthenol, glycol stearate, ceteth-2, hydroxyethylcellulose, phenoxyethanol, methylparaben, propylparaben, citric acid, mica, titanium dioxide, iron oxide, fragrance, or any combination thereof.

One or more examples of a hair spray or hair gel may comprise a composition of the present disclosure and one or more of cyclomethicone, jojoba ester, dimethicone copolyol, nonfat dry milk, soy protein, stearic acid, capric/caprylic stearic triglyceride, jojoba oil, hybrid sunflower oil, cetearyl alcohol, glyceryl stearate, PEG-40 stearate, aloe vera gel, acrylates/C₁₀-30 alkyl acrylate crosspolymer, propylene glycol, tocopheryl acetate, methylparaben, propylparaben, fragrance, or any combination thereof.

### Cosmetics

Cosmetics are formulations that may be used for altering or improving one's physical appearance. Illustrative cosmetics include, but are not limited to, lipstick, blush, mascara, foundation, eyeliner, and the like. Forms of cosmetics may include, for example, emulsions, creams, gels, dispersions, sticks, and the like. Suitable emulsions within cosmetics may include oil-in-water or water-in-oil emulsions.

A reaction product of a dextrin or dextran and a fatty acid or a fatty ester, as specified above, in combination with a neutral surfactant or a zwitterionic surfactant, preferably a neutral surfactant may be present in various types of cosmetics in which surfactants may be used. The compositions of the disclosure herein may replace a surfactant used in cosmetic or be used in combination with a surfactant already present in a cosmetic. Within a cosmetic, compositions comprising the reaction products and a surfactant may be present in an amount of about 0.01 wt. % to about 20 wt. % of the adjuvant composition as a whole, or about 0.1 wt. % to about 10 wt. %, or about 1 wt. % to about 15 wt. %, or about 5 wt. % to about 20 wt. %.

Examples of suitable additional components that may be present in cosmetics include, but are not limited to, other surfactants, perfumes, preservatives, coloring materials, UV absorbers, moisture-retaining agents, emulsifiers, gelling agents, oils, thickening agents, foam stabilizers, viscosity builders, preservatives, sequestrates, antioxidants, suspending agents, proteins, fragrances, sunscreens, botanical extracts, essential oils, fats (e.g., shea butter, mango seed butter, and cacao seed butter), fatty acids, fatty esters, fatty alcohols, biocides, soaps, preservatives, acids, bases, buffers, chelating agents, thickeners, vitamins, waxes (e.g., myristyl myristate, Camellia Sinensis leaf extract, jojoba, sunflower seed, carnauba wax, candelilla wax, and beeswax), and the like, including any combination thereof. Some additional examples of components that may be present in cosmetics may include, for example, higher fatty alcohols such as cetyl alcohol, stearyl alcohol and behenyl alcohol; higher fatty acid including caprylic/capric triglyceride, lauric acid, myristic acid, palmitic acid and stearic acid; hydrocarbons including ceresin; natural oils including meadowfoam seed oil, sunflower seed oil, macadamia seed oil, green tea seed oil, ginger oil, ginseng oil, coconut oil, olive oil and camellia oil; esters including phytosteryl/octyldodecyl lauroyl glutamate, isostearyl isostearate, methylheptyl isostearate, dicaprylyl carbonate and isopropyl palmitate; ethers including dicaprylyl ether; silicone oils including dimethicone, cyclopentasiloxane, cyclohexasiloxane, phenyltrimethicone, trisiloxane and methyltrimethicone; and hydrocarbons including squalane. Examples of suitable additional components will be familiar to one having ordinary skill in the art. Other surfactants that may be present in cosmetics are not particularly limited and may be any one or a combination of cationic, anionic, neutral, or zwitterionic surfactants. The reaction products may replace at least a portion of one or more existing surfactants in a cosmetic or supplement a quantity of one or more existing surfactants in a cosmetic. Cosmetics of the present disclosure may be formulated in any suitable form including, sticks, creams, powders, gels, and the like.

Some examples of cosmetics comprising a reaction product of the present disclosure may include compositions containing another surfactant. Alternately, the neutral or zwitterionic surfactant introduced with the reaction product may constitute the entirety of the surfactants in the cosmetics. Other surfactants may be present in an amount up to about 20 wt. % of the cosmetics.

Some examples of the cosmetics may comprise an oil-in-water emulsion. Other examples of the cosmetics may comprise a water-in-oil emulsion. Other suitable forms of cosmetics may comprise a powder, stick, suspension, gel, cream, or similar formulation

Some examples of the cosmetics may include the reaction product of the saccharide polymer in combination with one or more inorganic salts, such as potassium chloride, sodium chloride, or the like. A ratio of reaction product to inorganic salts may range from about 1:99 to 99:1 on a weight basis.

Some examples of the cosmetics may include the reaction product of the saccharide polymer in combination with a moisturizer.

Some examples of the cosmetics may include the reaction product of the saccharide polymer in combination with a coloring material.

Some examples of the cosmetics may include the reaction product of the saccharide polymer in combination with a sunscreen.

Some examples of the cosmetics may include the reaction product of the saccharide polymer in combination with one or more oils (including essential oils), waxes, or fats. Some examples of the cosmetics may be substantially oil-free.

### Deodorants and Antiperspirants

Deodorants and antiperspirants are formulations that may be utilized for controlling body odor. Deodorants and antiperspirants of the present disclosure may be formulated in stick form, gel form, powder form or aerosolizable form.

A reaction product of a dextrin or dextran and a fatty acid or a fatty ester, as specified above, in combination with a neutral surfactant or a zwitterionic surfactant may be present in deodorants and antiperspirants in which surfactants may be used. The compositions of the disclosure herein may replace a surfactant used in a deodorant or antiperspirant or be used in combination with a surfactant already present in a deodorant or antiperspirant. Within a deodorant or antiperspirant, compositions comprising the reaction products and a surfactant may be present in an amount of about 0.01 wt. % to about 20 wt. % of the deodorant or antiperspirant composition as a whole, or about 0.1 wt. % to about 10 wt. %, or about 1 wt. % to about 15 wt. %, or about 5 wt. % to about 20 wt. %.

Examples of suitable additional components that may be present in deodorants or antiperspirants disclosed herein include, but are not limited to, other surfactants, aluminum salts (e.g., alum, aluminum chloride, aluminum chlorohydrate, aluminum-zirconium compounds, aluminum-zirconium tetrachlorohydrex gly, and aluminum-zirconium tetrachlorohydrex gly), anti-bacterial agents, parabens, alcohols, propylene glycol, hexamethylenetetramine, acids, bases, buffers, chelating agents, perfumes, preservatives, coloring materials, moisture-absorbing agents (dessicants), emulsifiers, gelling agents, oils, thickening agents, foam stabilizers, viscosity builders, sequestrates, antioxidants, suspending agents, fragrances, essential oils, fats, fatty acids, fatty esters, fatty alcohols, waxes, and the like, including any combination thereof. Examples of suitable additional components will be familiar to one having ordinary skill in the art. Other surfactants that may be present in the deodorants and antiperspirants are not particularly limited and may be any one or a combination of cationic, anionic, neutral, or zwitterionic surfactants. The reaction products may replace at least a portion of one or more existing surfactants in a deodorant or antiperspirant or supplement a quantity of one or more existing surfactants in a deodorant or antiperspirant. Deodorants and antiperspirants of the present disclosure may be formulated in any suitable form including, sticks, creams, powders, gels, and the like.

Some examples of deodorants and antiperspirants comprising a reaction product of the present disclosure may include compositions containing another surfactant. Alternately, the neutral or zwitterionic surfactant introduced with the reaction product may constitute the entirety of the surfactants in the deodorants and antiperspirants. Other surfactants may be present in an amount up to about 10 wt. % of the deodorants and antiperspirants.

Some examples of the deodorants and antiperspirants may include the reaction product of the saccharide polymer in combination with one or more inorganic salts, such as potassium chloride, sodium chloride, or the like. A ratio of reaction product to inorganic salts may range from about 1:99 to 99:1 on a weight basis.

Some examples of the deodorants and antiperspirants may include the reaction product of the saccharide polymer in combination with an aluminum salt that provides deodorant action.

Some examples of the deodorants and antiperspirants may include the reaction product of the saccharide polymer in combination with an anti-bacterial agent.

### Froth Floatation Applications

The compositions of the present disclosure comprising a reaction product of a dextrin compound, a dextran, or any combination thereof with a fatty acid or a fatty ester may also find exemplary uses and formulations outside the personal care space as well. The reaction products disclosed herein may be incorporated in applications in which metal sequestration from a fluid is needed, such as within froth floatation processes. Froth floatation processes may be conducted in various instances, such as mining runoff or water treatment. In such applications, the compositions of the present disclosure may replace a surfactant used in froth floatation or be used in combination with a surfactant already present in froth floatation process. Within a given froth floatation process, compositions comprising the reaction products and a surfactant may be present in an amount of about 0.01 wt. % to about 20 wt. % of a fluid as a whole, or about 0.1 wt. % to about 10 wt. %, or about 1 wt. % to about 15 wt. %, or about 5 wt. % to about 20 wt. %.

In some examples, the compositions of the present disclosure may be utilized in roughers and cleaner circuits to promote clay dispersion, water conditioning, additive enhancement and/or emulsification of metal suppressants such as Mn and Fe. Any conventional frothing agent may be utilized in combination with the compositions disclosed herein. Suitable frothing agents and details concerning frothing agents will be familiar to one having ordinary skill in the art.

Embodiments disclosed herein include:

A. Consumer or industrial products comprising a saccharide polymer reaction product with a fatty acid. The products comprise: a carrier phase, a neutral surfactant or a reaction product thereof combined with the carrier phase, and a reaction product of a saccharide polymer and a fatty acid combined with the carrier phase, the saccharide polymer comprising a dextran, a dextrin compound, or any combination thereof. The reaction product of the saccharide polymer and the fatty acid is present at a concentration effective to lower a surface tension of the neutral surfactant.

A1. The composition of A, wherein the saccharide polymer comprises dextran.

A2. The composition of A, wherein the saccharide polymer comprises a dextrin compound, preferably maltodextrin.

Embodiments A, A1 and A2 may have one or more of the following additional elements in any combination.

Element 1: wherein the saccharide polymer comprises a dextrin compound and the dextrin compound comprises a maltodextrin.

Element 2: wherein the maltodextrin has a dextrose equivalent value of about 3 to about 20.

Element 3: wherein the maltodextrin has a dextrose equivalent value of about 4.5 to about 7.0.

Element 4: wherein the maltodextrin has a dextrose equivalent value of about 9.0 to about 12.0.

Element 5: wherein the fatty acid comprises about 4 to about 30 carbon atoms.

Element 6: wherein the fatty acid comprises at least one fatty acid selected from the group consisting of butyric acid, valeric acid, caproic acid, enanthic acid, caprylic acid, pelabonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, trioscylic acid, lignoceric acid, pentacosylic acid, cerotic acid, carboceric acid, montanic acid, nonacosylic acid, melissic acid, crotonic acid, cervonic acid, linoleic acid, linolelaidic acid, linolenic acid, arachidonic acid, docosatetraenoic acid, myristoleic acid, palmitoleic acid, sappenic acid, vaccenic acid, paullinic acid, oleic acid, pinolenic acid, stearidonic acid, eleostearic acid, elaidic acid, gondoic acid, gadoleic acid, erucic acid, eicosenoic acid, eicosadiencoic acid, eicosatrienoic acid, eicosatetraenoic acid, docosadienoic acid, nervonic acid, mead acid, adrenic acid, and any combination thereof.

Element 7: wherein the neutral surfactant comprises cocamide diethanolamine or a reaction product thereof, or wherein the neutral surfactant comprises cocamide diethanolamine or a reaction product thereof.

Element 7A: wherein the reaction product is formed in the presence of the neutral surfactant.

Element 8: wherein a molar ratio of fatty acid to dextrin in the reaction product is about 0.2 or above on a basis of moles_{fatty acid}:moles_{glucose monomers}.

Element 8A: wherein a molar ratio of fatty acid to dextrin in the reaction product is about 0.05 or above on a basis of moles_{fatty acid}:moles_{glucose monomers}.

Element 9: wherein a molar ratio of fatty acid to dextrin in the reaction product is about 0.35 or above on a basis of moles_{fatty acid}:moles_{glucose monomers}.

Element 10: wherein the reaction product of the saccharide polymer is obtained in the presence of water and a hydroxide base.

Element 11: wherein the reaction product of the saccharide polymer comprises a fatty ester reaction product.

Element 12: wherein the consumer or industrial product is foamed or foamable, emulsified, in stick form, or is formulated as a cream or gel.

By way of non-limiting example, exemplary combinations applicable to A, A1, and A2 include, but are not limited to: 1, and 2, 3 or 4; 1 and 5; 1 and 6; 1, and 7 or 7A; 1, and 8, 8A or 9; 1 and 11; 1 and 12; 5, and 7 or 7A; 5, and 8, 8A or 9; 5 and 11; 5 and 12; 7 or 7A, and 8, 8A or 9; 8, 7 or 7A, and 11; and 7 or 7A, and 12.

Additional embodiments disclosed herein include:

A′: Consumer or industrial products. The products comprise: a carrier phase; a neutral surfactant or a reaction product thereof combined with the carrier phase; and a reaction product of a saccharide polymer and a fatty acid or a fatty ester combined with the carrier phase, the saccharide polymer comprising a dextran, a dextrin compound, or any combination thereof, and the fatty acid comprising about 50 wt. % or above of one or more straight-chain fatty acids.

A1′. The composition of A, wherein the saccharide polymer comprises dextran.

A2′. The composition of A′, wherein the saccharide polymer comprises a dextrin compound, preferably maltodextrin.

Embodiments A′, A1′ and A2′ may have one or more of the following additional elements in any combination.

Element 1′: wherein the reaction product of the saccharide polymer and the fatty acid or the fatty ester is present at a concentration effective to lower a surface tension of the neutral surfactant in an aqueous fluid.

Element 2′: wherein the saccharide polymer comprises a dextrin compound and the dextrin compound comprises a maltodextrin.

Element 3′: wherein the maltodextrin has a dextrose equivalent value of about 3 to about 25.

Element 4′: wherein the fatty acid or a fatty acid component obtainable from the fatty ester comprises about 4 to about 30 carbon atoms.

Element 5′: wherein the fatty acid or a fatty acid component obtainable from the fatty ester consists of one or more straight-chain fatty acids comprising about 4 to about 30 carbon atoms.

Element 6′: wherein the fatty acid or a fatty acid component obtainable from the fatty ester comprises at least one fatty acid selected from the group consisting of butyric acid, valeric acid, caproic acid, enanthic acid, caprylic acid, pelabonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, trioscylic acid, lignoceric acid, pentacosylic acid, cerotic acid, carboceric acid, montanic acid, nonacosylic acid, melissic acid, crotonic acid, cervonic acid, linoleic acid, linolelaidic acid, linolenic acid, arachidonic acid, docosatetraenoic acid, myristoleic acid, palmitoleic acid, sappenic acid, vaccenic acid, paullinic acid, oleic acid, pinolenic acid, stearidonic acid, eleostearic acid, elaidic acid, gondoic acid, gadoleic acid, erucic acid, eicosenoic acid, eicosadiencoic acid, eicosatrienoic acid, eicosatetraenoic acid, docosadienoic acid, nervonic acid, mead acid, adrenic acid, and any combination thereof.

Element 7′: wherein the neutral surfactant comprises a fatty acid alkanolamide or a reaction product thereof.

Element 8′: wherein the fatty acid alkanolamide comprises a compound selected from the group consisting of cocamide diethanolamine, cocamide monoethanolamine, cocamide diisopropanolamine, and any combination thereof.

Element 9′: wherein a molar ratio of fatty acid to saccharide polymer in the reaction product is about 0.2 or above on a basis of moles_{fatty acid}:moles_{glucose monomers}.

Element 10′: wherein a molar ratio of fatty acid to saccharide polymer in the reaction product ranges from about 0.2 to about 0.8 on a basis of moles_{fatty acid}:moles_{glucose monomers}.

Element 11′: wherein the reaction product of the saccharide polymer is obtained in the presence of water and a hydroxide base.

Element 12′: wherein the reaction product of the saccharide polymer comprises a fatty ester saccharide polymer reaction product.

Element 13′: wherein the consumer or industrial product is foamed or foamable, or wherein the consumer or industrial product is emulsified, or wherein the consumer or industrial product is in a stick form, or wherein the consumer or industrial product is formulated as a cream or a gel.

Element 14′: wherein the consumer or industrial product further comprises at least one fatty acid carboxylate.

Element 15′: wherein the consumer or industrial product further comprises at least one zwitterionic surfactant.

Element 16′: wherein the reaction product is formed from a fatty ester, and the consumer or industrial product further comprises glycerol.

By way of non-limiting example, exemplary combinations applicable to A′, A1′, or A2′ include, but are not limited to, 1′ and 2′; 1′-3′; 1′ and 4′; 1′ and 5′; 1′ and 6′; 1′ and 7′; 1′ and 8′; 1′ and 9′; 1′ and 10′; 1′ and 11′; 1′ and 12′; 1′ and 13′; 1′ and 14′; 1′ and 15′; 1′ and 16′; 2′ or 3′, and 4′; 2′ or 3′, and 5′; 2′ or 3′, and 6′; 2′ or 3′, and 7′; 2′ or 3′, and 8′; 2′ or 3′, and 9′; 2′ or 3′, and 10′; 2′ or 3′, and 11′; 2′ or 3′, and 12′; 2′ or 3′, and 13′; 2′ or 3′, and 14′; 2′ or 3′, and 15′; 2′ or 3′, and 16′; 4′ or 5′, and 7′; 4′ or 5′, and 8′; 4′ or 5′, and 9′; 4′ or 5′, and 10′; 4′ or 5′, and 11′; 4′ or 5′, and 12′; 4′ or 5′, and 13′; 4′ or 5′, and 14′; 4′ or 5′, and 15′; 4′ or 5′, and 16′; 6′ and 7′; 6′ and 8′; 6′ and 9′; 6′ and 10′; 6′ and 11′; 6′ and 12′; 6′ and 13′; 6′ and 14′; 6′ and 15′; 6′ and 16′; 7′ or 8′, and 9′; 7′ or 8′, and 10′; 7′ or 8′, and 11′; 7′ or 8′, and 12′; 7′ or 8′, and 13′; 7′ or 8′, and 14′; 7′ or 8′, and 15′; 7′ or 8′, and 16′; 9′ or 10′, and 11′; 9′ or 10′, and 12′; 9′ or 10′, and 13′; 9′ or 10′, and 14′; 9′ or 10′, and 15′; 9′ or 10′, and 16′; 11′ and 12′; 11′ and 13′; 11′ and 14′; 11′ and 15′; 11′ and 16′; 12′ and 13′; 12′ and 14′; 12′ and 15′; 12′ and 16′; 13′ and 14′; 13′ and 15′; 13′ and 16′; 14′ and 15′; 14′ and 16′; and 15′ and 16′.

To facilitate a better understanding of the disclosure herein, the following examples of various representative embodiments are given. In no way should the following examples be read to limit, or to define, the scope of the invention.

### EXAMPLES

Comparative Example 1: Acid-Catalyzed Reaction of Maltodextrin with Lauric Acid. A solution containing 10 wt. % maltodextrin (MALTRIN M100, DE=9.0-12.0, 30% active solution) and 6.18 wt. % lauric acid was prepared in DMSO. Five drops of phosphoric acid were added, and the reaction mixture was heated at 110ºC for 3 hours. The reaction product was precipitated by adding 3 volumes of isopropyl alcohol, and a white precipitate was collected by decantation and dried. The product was characterized by FTIR and 1H NMR. The spectral characterization was consistent with conversion of maltodextrin into a reaction product.

For surface tension measurements (Table 2), the isolated reaction product was redissolved at a concentration of 13.17 wt. % in the presence of 5 wt. % cocamide diethanolamine (CocoDEA) and 6 wt. % sodium dodecylbenzene sulfonate (SDDBS).

Comparative Example 2: Acid Chloride-Based Reaction of Maltodextrin. A solution containing 10 wt. % maltodextrin (MALTRIN M100, DE=9.0-12.0, 30% active solution) and 6.75 wt. % lauroyl chloride was prepared in formamide. A few drops of phosphoric acid were added, and the reaction mixture was heated at 105ºC for 2 hours. The reaction product was precipitated by adding 3 volumes of isopropyl alcohol, and an amber tar-like fluid was obtained. The product was characterized by FTIR and 1H NMR. The spectral characterization was consistent with conversion of maltodextrin into a reaction product.

For surface tension measurements (Table 2), the isolated reaction product was redissolved at a concentration of 13.17 wt. % in the presence of 5 wt. % cocamide diethanolamine (CocoDEA) and 6 wt. % sodium dodecylbenzene sulfonate (SDDBS).

Example 1A: General Procedure for Preparation of Reaction Products of Maltodextrin Under Basic Conditions. 296.25 g water, 25.00 g cocamide diethanolamine (CocoDEA), and 10.00 g KOH (45% active solution) were combined. The reaction mixture was mechanically stirred and heated to 65ºC. Thereafter, 18.75 g fatty acid and 150.0 g maltodextrin (MALTRIN M100, Grain Processing Corporation, Muscatine, Iowa; DE=9.0-12.0) as a 30% active solution were added to the reaction mixture. Once the maltodextrin dissolved, heating was discontinued and stirring was conducted until the reaction mixture reached room temperature. Reaction products were used without further processing below. Table 1A shows the maltodextrin reaction products synthesized as above and tested in the subsequent examples. Caprylic acid is synonymous with octanoic acid, lauric acid is synonymous with dodecanoic acid, and stearic acid is synonymous with octadecanoic acid.

TABLE 1A

| | | Molar Ratio |
|---|---|---|
| | | Fatty Acid:Maltodextrin |
| Sample | Fatty Acid | (as Glucose Monomer) |
| A | Butyric Acid | 0.77 |
| B | Caprylic Acid | 0.47 |
| C | Lauric Acid | 0.34 |
| D | Stearic Acid | 0.24 |

The general synthetic procedure was followed for all but Sample A. For Sample A, 27.5 g KOH (45% active) and 278.75 g water were used, and the other reaction parameters remained the same. The calculated molar ratios assume that the entirety of the maltodextrin has the molecular weight of glucose (180.16 g/mol) less the molecular weight of water (18.02 g/mol)=162.14 g/mol.

Example 1B: Alternative Procedure for Preparation of Reaction Products of Maltodextrin Under Basic Conditions. A solution containing 10 wt. % maltodextrin (MALTRIN M100, DE=9.0-12.0, 30% active solution), 6.18 wt. % lauric acid, and 1.73 wt. % KOH was prepared in water. The reaction mixture was then heated at 65ºC for 30 minutes. The reaction product was precipitated by adding 3 volumes of isopropyl alcohol, and a white precipitate was collected by decantation and dried. The product was characterized by FTIR and 1H NMR. The spectral characterization was consistent with conversion of maltodextrin into a reaction product. Other fatty acids may be reacted similarly.

For surface tension measurements (Table 2), the isolated reaction product was redissolved at a concentration of 13.17 wt. % in the presence of 5 wt. % cocamide diethanolamine (CocoDEA) and 6 wt. % sodium dodecylbenzene sulfonate (SDDBS). As shown in Table 2, similar surface tension performance was realized between the reaction products of Example 1A and Comparative Examples 1 and 2.

Example 1C: General Procedure for Preparation of Reaction Products of Maltodextrin Under Basic Conditions Starting From a Fatty Ester. 25.00 g cocamide diethanolamine (CocoDEA) and 10.00 g KOH (45% active solution) were combined in water. The reaction mixture was mechanically stirred and heated to 65ºC. Thereafter, soybean oil and 150.0 g maltodextrin (MALTRIN M100, Grain Processing Corporation, Muscatine, Iowa; DE=9.0-12.0) as a 30% active solution were added to the reaction mixture. The amount of soybean oil was selected to provide a HLB of either 12 or 16 upon formation of a reaction product. The amount of water was selected to provide a surfactant concentration of 5 wt. %, a fatty ester (oil) concentration of 2.5 wt. %, and a maltodextrin concentration of 10 wt. %, based on all reaction components. Once the maltodextrin dissolved, heating was discontinued and stirring was conducted until the reaction mixture reached room temperature. The resulting aqueous phase containing the reaction products was used without further processing for the additional testing below. Dextran reaction products may be formed using a similar procedure.

Example 2: General Procedure for Preparation of Reaction Products of Dextran Under Basic Conditions. Reaction products were formed from dextran in a similar manner to that described above for maltodextrin. The dextran had a molecular weight of 500,000 and an activity level of 9% within a solution thereof. Table 1B shows the dextran reaction products synthesized as above and tested in the subsequent examples. Caprylic acid is synonymous with octanoic acid, lauric acid is synonymous with dodecanoic acid, palmitic acid is synonymous with hexadecanoic acid, and stearic acid is synonymous with octadecanoic acid.

TABLE 1B

| | | Weight Ratio Fatty | Molar Ratio |
|---|---|---|---|
| | | Acid:Weight | Fatty Acid:Dextran (as |
| Sample | Fatty Acid | Dextran | Glucose Monomer) |
| E1 | Caprylic Acid | 1:10 | 0.11 |
| E2 | Caprylic Acid | 1:5 | 0.22 |
| E3 | Caprylic Acid | 1:2 | 0.57 |
| E4 | Caprylic Acid | 1:1 | 1.13 |
| F1 | Lauric Acid | 1:10 | 0.081 |
| F2 | Lauric Acid | 1:5 | 0.16 |
| F3 | Lauric Acid | 1:2 | 0.41 |
| F4 | Lauric Acid | 1:1 | 0.81 |
| G1 | Palmitic Acid | 1:10 | 0.063 |
| G2 | Palmitic Acid | 1:5 | 0.13 |
| G3 | Palmitic Acid | 1:2 | 0.32 |
| G4 | Palmitic Acid | 1:1 | 0.63 |
| H1 | Stearic Acid | 1:10 | 0.057 |
| H2 | Stearic Acid | 1:5 | 0.11 |
| H3 | Stearic Acid | 1:2 | 0.28 |
| H4 | Stearic Acid | 1:1 | 0.57 |

Characterization of Comparative Examples 1 and 2 in Relation to Example 1B. Table 2 summarizes the surface tension values for the reaction products of Comparative Examples 1 and 2 and the reaction product of Example 1B (alternative preparation under basic conditions) at a concentration of 1 gpt (gallons per thousand gallons), in comparison to control samples containing 5 wt. % CocoDEA or 5 wt. % CocoDEA/6 wt. % SDDBS. Surface tension (ST) measurements were made using a Bolin Scientific Tensiometer at room temperature. Intrafacial tension (IFT) measurements were performed using a hook needle syringe to form a drop of oil in water.

TABLE 2

| | | Surface Tension |
|---|---|---|
| | | at 1 gpt |
| Entry | Sample | (dynes/cm) |
| 1 | Control | 31.26 |
| | (5 wt. % CocoDEA/ | |
| | 6 wt. % SDDBS) | |
| 2 | Control | 34.49 |
| | (5 wt. % CocoDEA) | |
| 3 | Comparative | 31.77 |
| | Example 1 | |
| 4 | Comparative | 31.48 |
| | Example 2 | |
| 5 | Example 1B | 31.51 |

The control samples and comparative/experimental samples contained identical concentrations of CocoDEA or CocoDEA/SDDBS. As shown, the reaction product prepared under basic conditions (Entry 5) afforded similar performance to that obtained under acidic conditions (Entries 3 and 4). In each case, the surface tension was similar to that of the surfactant-only CocoDEA/SDDBS control (Entry 1). The surface tension values decreased by about 10% relative to a CocoDEA-only control (Entry 2). This surprising result is further elaborated upon below.

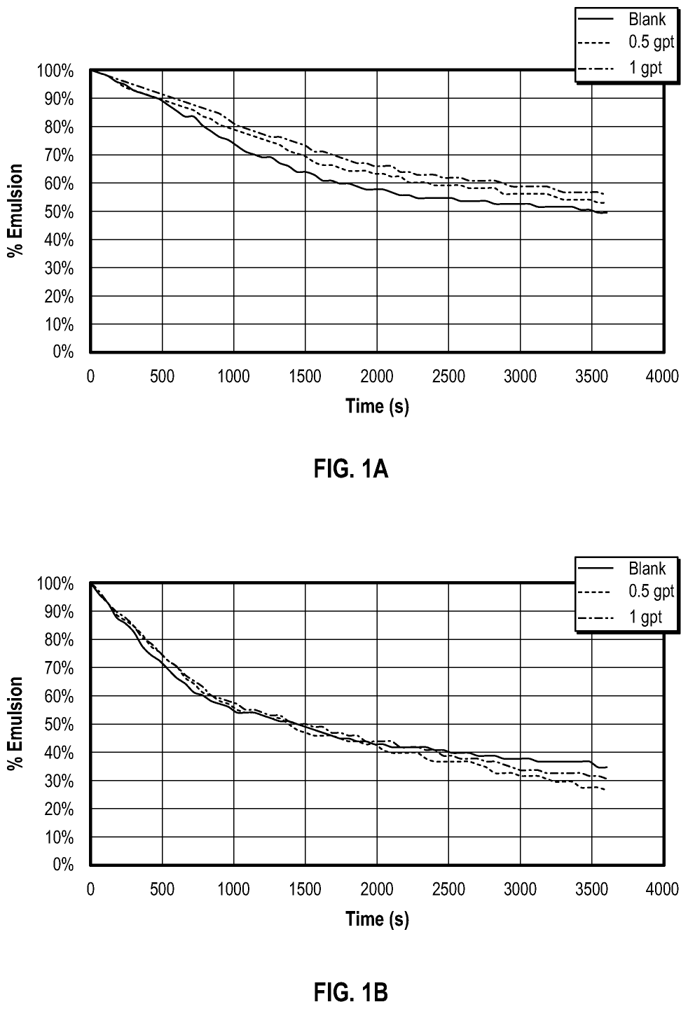

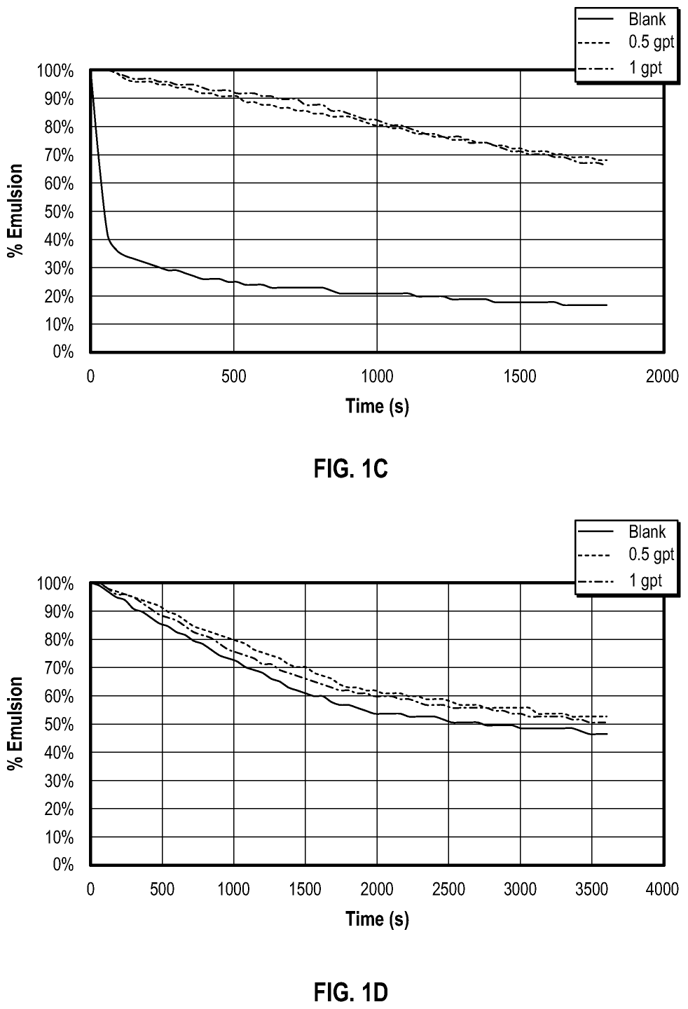

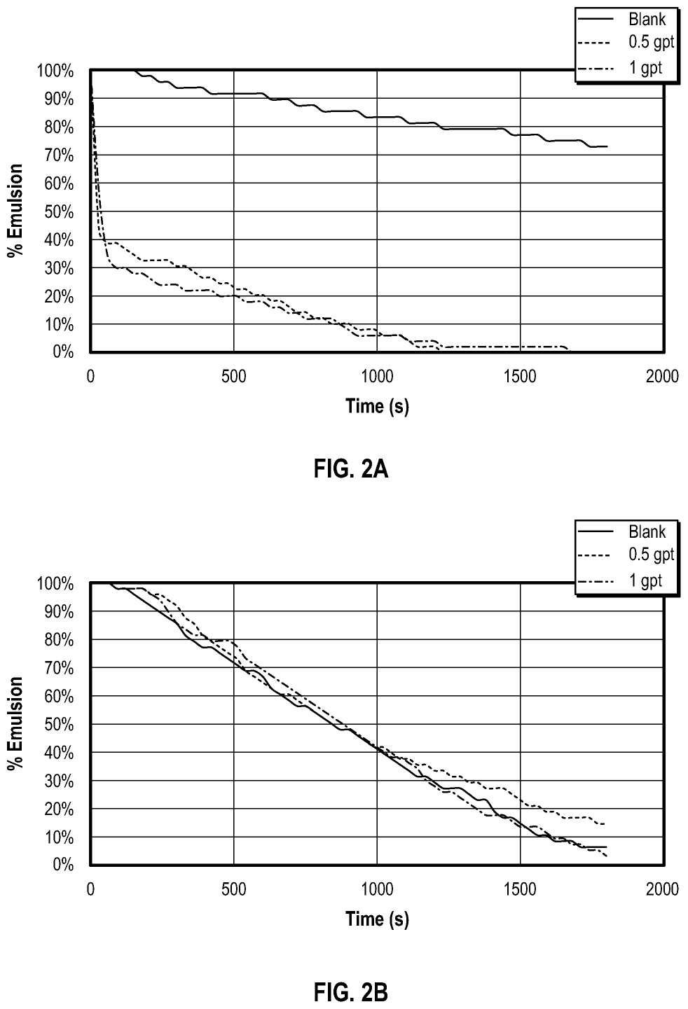

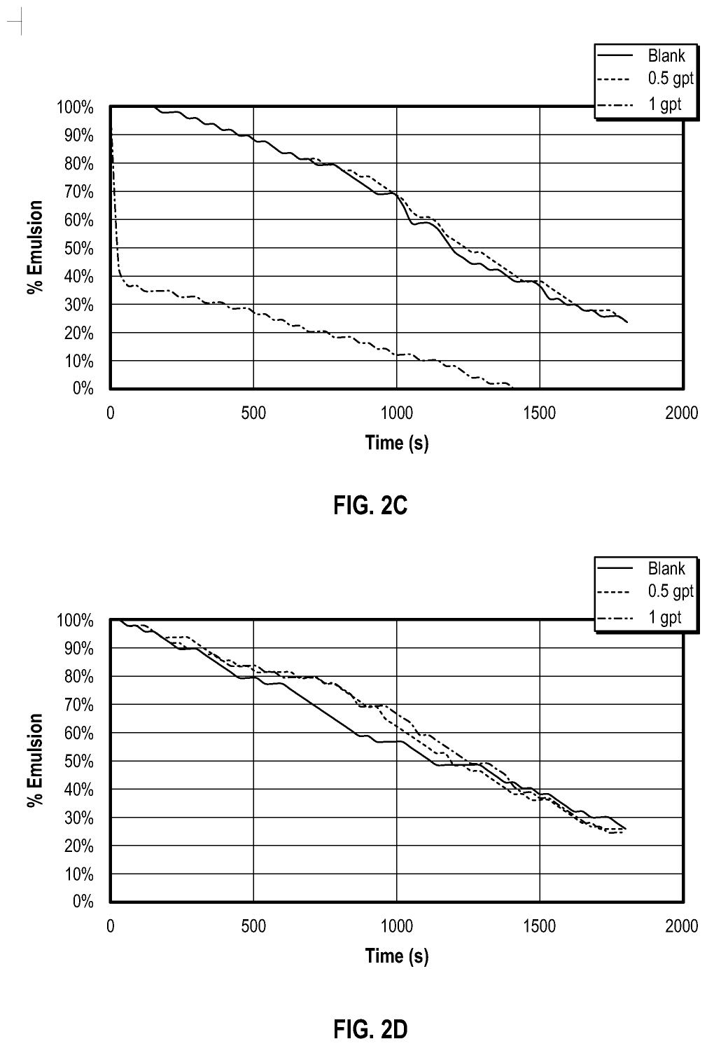

Emulsion Performance of Dextrin Reaction Products. Each reaction product prepared as above in Example 1A was formulated at 0.5 gpt (gallons per thousand gallons) and 1 gpt and combined with Terero oil or Wolfcamp A Oil. Terero oil is an emulsifying oil, and Wolfcamp A oil is a non-emulsifying oil. The mixture of each oil was then emulsified and the extent of emulsification was followed as a function of time in comparison to a blank. The blank comprised each oil without any additional emulsifiers. Emulsification was performed at room temperature by shaking 50 mL of sample and 50 ml of oil by hand for 60 seconds at a rate of about 2 shakes per second. The emulsions were immediately poured into a graduated cylinder and time-lapse photography was used to record the level of the water layer, the oil layer, and the remaining emulsion layer. For Wolfcamp A oil, the oil layer and the water layer were assumed equal, since the oil layer was difficult to differentiate from the emulsion layer. FIGS. 1A-1D show plots of percent emulsification as a function of time for Terero oil emulsified with Samples A-D, respectively. FIGS. 2A-2D show plots of percent emulsification as a function of time for Wolfcamp A oil emulsified with Samples A-D, respectively.

Both oils were initially emulsified in the presence of the maltodextrin reaction products, but the emulsions broke over time, albeit at different rates. Terero oil usually changed its emulsification behavior only slightly in the presence of the reaction products formed from carboxylic acids having varying chain lengths. With the non-emulsifying Wolfcamp A oil, in contrast, the maltodextrin reaction products sometimes afforded faster emulsion breaking than did the control. The results suggest that the maltodextrin reaction products, particularly the specific fatty acid used for functionalization and the amount of reaction product present, may alter the break properties of the non-emulsifying Wolfcamp oil itself to varying extents. The differing performance may arise from variation of the hydrophilic-lipophilic balance. Moreover, the break properties may differ from that of CocoDEA alone, which afforded a near-complete break of Wolfcamp oil within about 30 minutes at 1 gpt (data not shown).

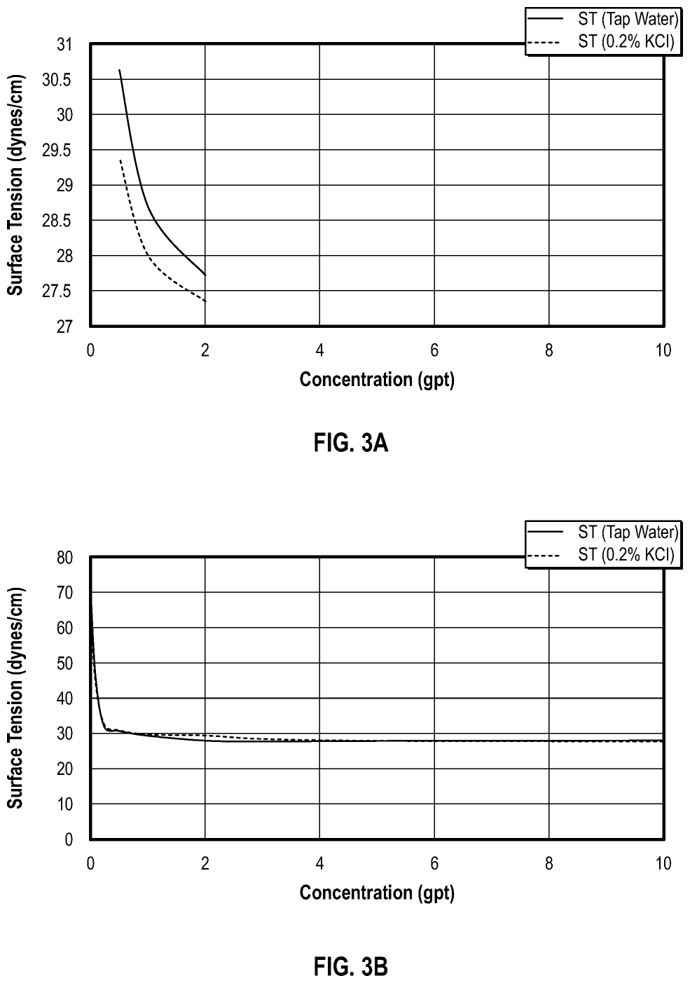

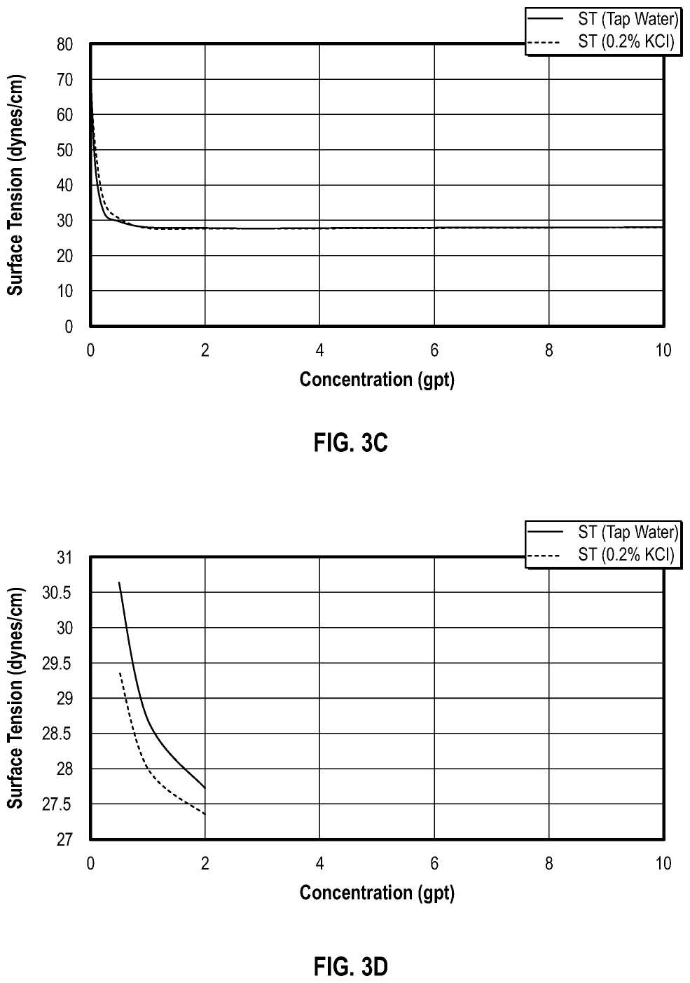

Fluid Properties of Dextrin Reaction Products. Critical micelle concentration (CMC) measurements and surface tension (ST) measurements were made using a Bolin Scientific Tensiometer at room temperature. FIGS. 3A-3D show plots of surface tension as a function of concentration for Samples A-D, respectively. As shown, Samples B and C reached a CMC at a reaction product concentration of about 0.5 gpt. The surface tension at the CMC was approximately 30 dynes/cm or slightly below. Samples A and D, in contrast, trended toward lower surface tension values, albeit at higher CMCs. As such, the emulsion performance measurements above were performed above the CMC for at least Samples B and C. The surface tension was slightly higher for 0.2 wt. % KCl compared to that obtained with tap water.

The surface tension performance of individual components of the reaction mixture used to produce Sample C were also compared against that of the reaction product itself. Measurements were made at 1 gpt and 2 gpt, as specified in Table 3 below.

TABLE 3

| | | ST at 1 gpt | ST at 2 gpt |
|---|---|---|---|
| Entry | Component | (dynes/cm) | (dynes/cm) |
| 1 | maltodextrin (30% active | 73.03 | 72.85 |
| | solution) | | |
| 2 | maltodextrin (30% active | n/d | 77.39 |
| | solution), 1.7% KOH (45% | | |
| | active solution) and 6.18% | | |
| | lauric acid (heated as | | |
| | above) | | |
| 3 | 10% maltodextrin (30% | 49.23 | 35.89 |
| | active solution), 5% | | |
| | CocoDEA neutral surfactant | | |
| | solution (heated as above) | | |
| 4 | 5% CocoDEA neutral | 34.49 | 31.93 |
| | surfactant solution (heated | | |
| | as above) | | |
| 5 | 5% CocoDEA neutral | 36.73 | 35.00 |
| | surfactant solution | | |
| | containing 2% KOH (45% | | |
| | active solution) (heated as | | |
| | above) | | |
| 6 | 5% CocoDEA neutral | 39.88 | 32.99 |
| | surfactant solution | | |
| | containing 2.47% lauric | | |
| | acid (heated as above) | | |
| 7 | 5% CocoDEA neutral | 36.52 | 30.40 |
| | surfactant solution | | |
| | containing 2% KOH (45% | | |
| | active solution) and 3.75% | | |
| | lauric acid (heated as | | |
| | above) | | |
| 8 | Sample C | 28.84 | 28.59 |

As shown, the maltodextrin itself (Entry 1) afforded a very high surface tension in comparison to Sample C (Entry 8). In the absence of CocoDEA, the surface tension remained very high even when other components used to form the reaction product were present (Entry 2). 5 wt. % CocoDEA afforded a much lower surface tension (Entry 4), which increased in the presence of maltodextrin (Entry 3). When other components used to form the reaction mixture (except maltodextrin) were combined with 5 wt. % CocoDEA, the surface tension increased slightly (Entries 4-7) relative to the reaction product. In contrast, when all reaction components were present together in Sample C (Entry 8), the surface tension was lower than any other tested combination of reaction components. The decreased surface tension realized in the presence of the maltodextrin reaction product is particularly surprising, given that maltodextrin by itself increased the surface tension (Entries 3 and 4).

Tables 4 and 5 show the surface tension performance of Sample C at 1 gpt in water and CaCl₂)/water, respectively, at various pH values or CaCl₂) concentrations. The surface tension of Sample C slightly decreased further at more acidic pH values.

TABLE 4

| pH | Surface Tension |
|---|---|
| (H₂O) | (dynes/cm) |
| 1 | 25.05 |
| 4 | 27.11 |
| 7 | 28.25 |
| 10 | 28.25 |
| 14 | 28.64 |

TABLE 5

| CaCl₂ | Surface Tension |
|---|---|
| (wt. %) | (dynes/cm) |
| 0.2 | 28.06 |
| 2 | 29.27 |
| 10 | 27.17 |

Intrafacial tension (IFT) measurements were performed using a hook needle syringe to form a drop of oil in water. The measurements were made using tap water and Wolfcamp A oil and were evaluated after 61 hours of equilibration. Table 6 below summarizes the IFT performance of Sample C.

TABLE 6

| Concentration (gpt) | IFT (dynes/cm) |
|---|---|
| 0.5 | 8.57 |
| 1 | 7.51 |
| 2 | 5.48 |

The surface tension performance of the sample produced according to Example 1C is summarized in Table 7.

TABLE 7

| | | Surface Tension | | |
|---|---|---|---|---|
| | | (dynes/cm) | | |
| Entry | Sample Description | 2 gpt | 1 gpt | 0.5 gpt |
| 1 | Example 1C, HLB = 16 | 27.5 | 27.7 | 30.0 |
| 2 | Example 1C, HLB = 12 | 27.6 | 31.4 | 34.3 |
| 3 | Example 1C | 27.8 | 29.2 | 31.5 |
| | w/additional 10 wt. % | | | |
| | glycerol (based on | | | |
| | soybean oil) added | | | |
| | before heating, | | | |
| | HLB = 16 | | | |
| 4 | Example 1C | 30.6 | 33.2 | 34.9 |
| | w/additional 10 wt. % | | | |
| | glycerol (based on | | | |
| | soybean oil) added | | | |
| | before heating, | | | |
| | HLB = 12 | | | |
| 5 | Example 1C | 27.5 | 27.8 | 31.0 |
| | w/additional 10 wt. % | | | |
| | glycerol (based on | | | |
| | soybean oil) added | | | |
| | after heating, | | | |
| | HLB = 16 | | | |
| 6 | Example 1C | 32.4 | 32.9 | 35.3 |
| | w/additional 10 wt. % | | | |
| | glycerol (based on | | | |
| | soybean oil) added | | | |
| | after heating, | | | |
| | HLB = 12 | | | |
| 7 | 5 wt. % CocoDEA in | 31.9 | 34.5 | — |
| (control) | water (heated as in | | | |
| | Example 1C) | | | |
| 8 | 5 wt. % CocoDEA in 2% | 35.0 | 36.7 | — |
| (control) | aqueous KOH (45% | | | |
| | active solution) (heated | | | |
| | as in Example 1C) | | | |
| 9 | 2.5 wt. % soybean oil, | 30.0 | 33.0 | 41.4 |
| (control) | 10 wt. % maltodextrin | | | |
| | (30% active solution) | | | |
| | and 5 wt. % CocoDEA | | | |
| | in water, heated as | | | |
| | Example 1C | | | |
| 10 | 2.5 wt. % soybean oil, | 29.9 | 34.9 | 42.8 |
| (control) | 10 wt. % maltodextrin | | | |
| | (30% active solution) | | | |
| | and 5 wt. % CocoDEA | | | |
| | in water, no heating | | | |
| 11 | 10 wt. % maltodextrin | 35.9 | 49.2 | — |
| (control) | (30% active solution) | | | |
| | 5 wt. % CocoDEA in | | | |
| | water, heated as | | | |
| | Example 1C | | | |

As shown, soybean oil afforded a decrease in surface tension compared to CocoDEA alone (Entry 10). The reaction products afforded an additional decrease in surface tension (Entries 1 and 2). Introduction of additional glycerol over that released during alkaline hydrolysis of soybean oil resulted in slightly higher surface tension values in some cases. The lowering of the surface tension in the presence of the reaction products is therefore surprising in light of the tendency of the individual reaction components to raise or have minimal impact on surface tension values.

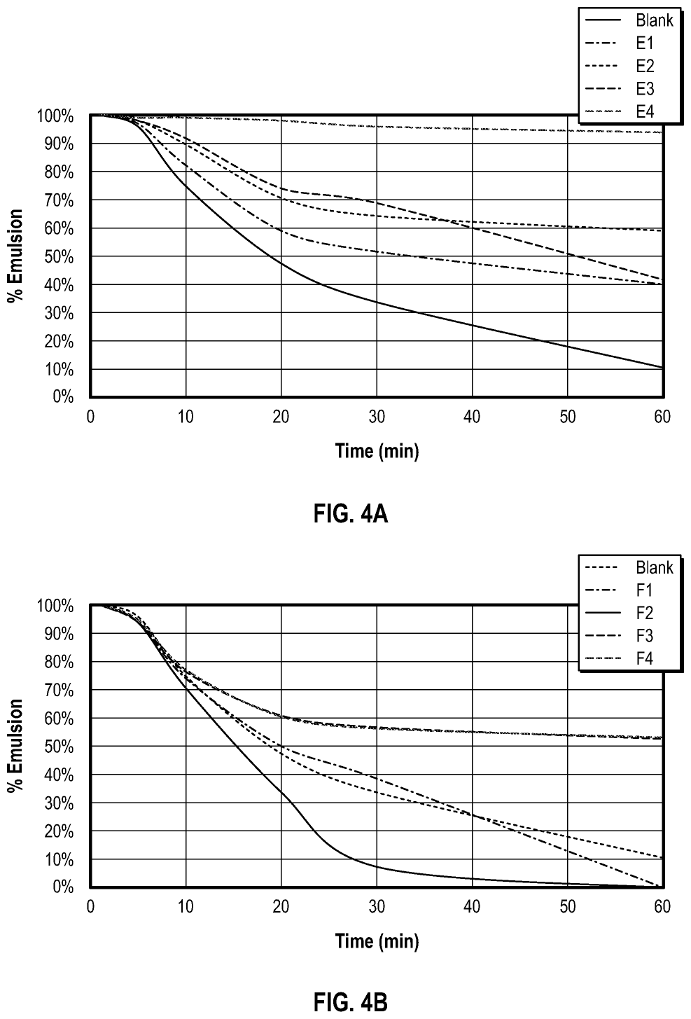

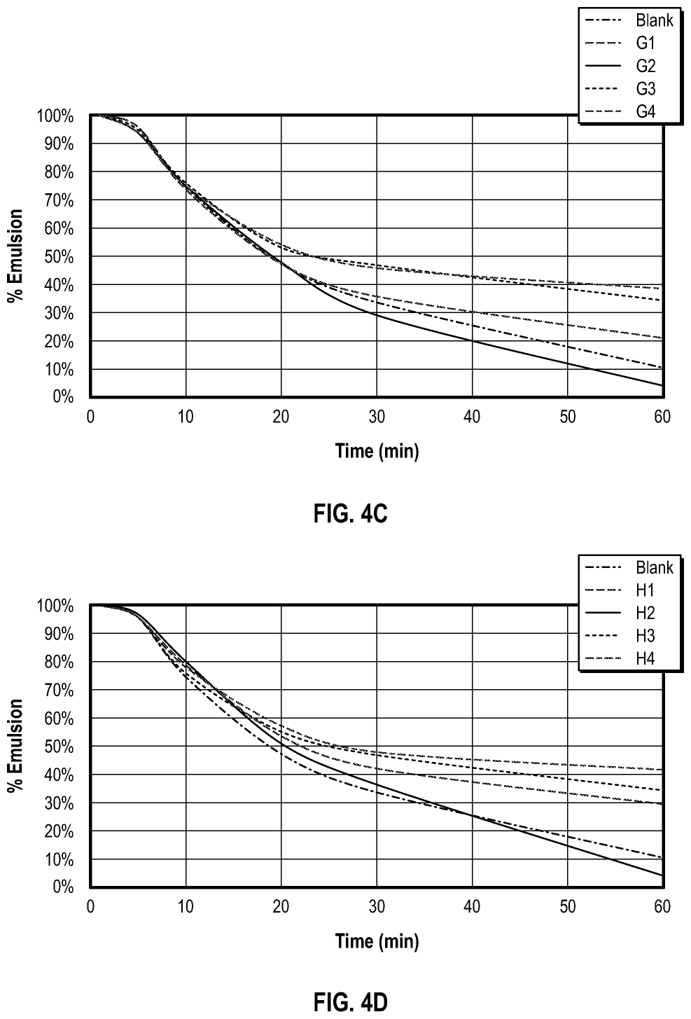

Emulsion Performance of Dextran Reaction Products. Each reaction product prepared as above was formulated at 1 gpt and combined with East Texas Hutchison oil #2. Each oil mixture was then emulsified, and the extent of emulsification was followed as a function of time in comparison to a blank. The blank comprised the oil without any additional emulsifiers. Emulsification was performed at room temperature by shaking 50 ml of sample and 50 ml of oil by hand for 60 seconds at a rate of about 2 shakes per second. The emulsions were immediately poured into a graduated cylinder and time-lapse photography was used to record the level of the water layer, the oil layer, and the remaining emulsion layer. FIGS. 4A-4D show plots of percent emulsification as a function of time for East Texas Hutchison #2 oil emulsified with Samples E1-E4, F1-F4, G1-G4 and H1-H4, respectively.

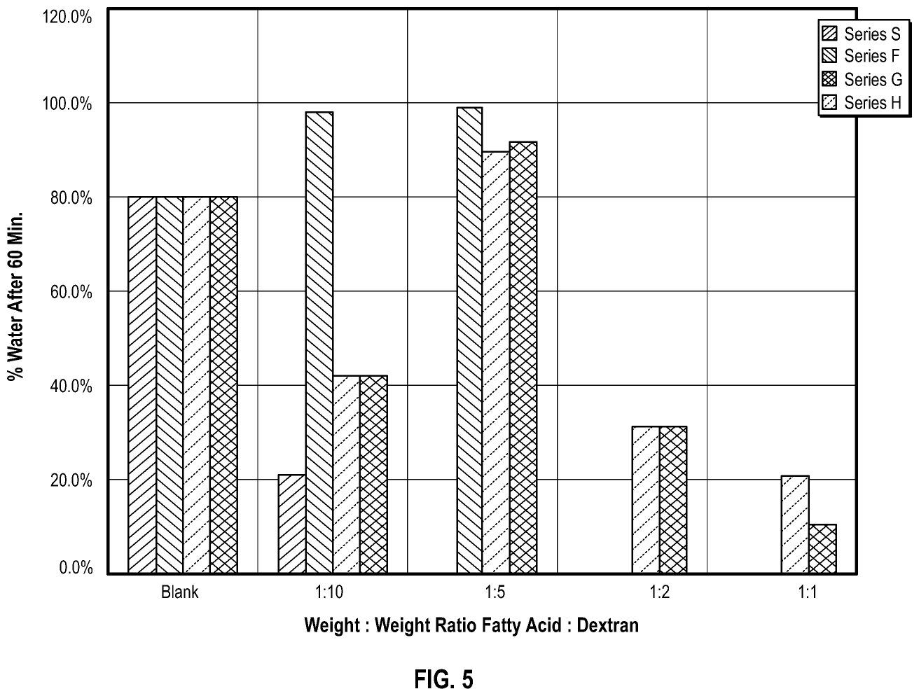

FIG. 5 shows a plot of percentage of de-emulsified water present after 60 minutes for each dextran reaction product at various weight ratios of fatty acid:dextran. As shown, the various dextran reaction products could promote emulsification or de-emulsification depending on the amount of fatty acid that was reacted with a given quantity of dextran. Series E samples (caprylic acid) afforded minimal emulsification. Series F samples (lauric acid) provided strong emulsification at weight ratios of 1:10 and 1:5, but emulsification decreased considerably at lower fatty acid loading. At weight ratios of 1:1 and 1:2 caprylic acid and lauric acid afforded little emulsification, but some degree of emulsification still occurred for palmitic and stearic acid (Series G and Series H samples) at these weight ratios. Overall, the strongest emulsification effects were observed at a weight ratio of 1:5 for all of the fatty acids except for caprylic acid (Series E samples).

Surface Tension of Dextran Reaction Products. The surface tension performance of the dextran reaction products was measured at 1 gpt and 2 gpt, as specified in Table 8 below.

TABLE 8

| | ST at 1 gpt | ST at 2 gpt |
|---|---|---|
| Sample | (dynes/cm) | (dynes/cm) |
| E1 | 34.61 | 30.25 |
| E2 | 34.07 | 29.15 |
| E3 | 32.39 | 28.87 |
| E4 | 31.95 | 29.00 |
| F1 | 30.49 | 28.75 |
| F2 | 29.93 | 27.93 |
| F3 | 31.34 | 27.65 |
| F4 | 68.20 | 54.71 |
| G1 | 33.82 | 28.25 |
| G2 | 28.77 | 27.46 |
| G3 | 31.51 | 28.53 |
| G4 | 45.76 | 38.04 |
| H1 | 34.66 | 28.75 |
| H2 | 33.06 | 28.27 |
| H3 | 38.07 | 32.13 |
| H4 | 49.97 | 40.98 |

As shown, all of the dextran reaction products were capable of lowering the surface tension of CocoDEA, at least at some concentrations and fatty acid loadings, in a manner similar to that provided by the maltodextrin reaction products described above. At the highest fatty acid loadings (samples F4, G4 and H4), the ability to lower the surface tension decreased considerably. Thus, the surface tension was tunable depending on the molecular weight of the fatty acid and the extent of fatty acid loading.

Example 3: Substitution of CocoDEA with Betaine Surfactant. Sample C′ was prepared in the same manner as Sample C above using the procedure of Example 1A and similar reagent proportions, except substituting a betaine (zwitterionic) surfactant (SOPALEX 360 BET) for CocoDEA and conducting the reaction at 50° C. Table 9 summarizes the surface tension of the reaction product in comparison to the betaine surfactant alone.

TABLE 9

| | ST at 1 gpt | ST at 2 gpt |
|---|---|---|
| Sample | (dynes/cm) | (dynes/cm) |
| Zwitterionic | 71.04 | 64.9 |
| Surfactant | | |
| Sample C′ | 66.27 | 55.55 |

Substitution of the betaine surfactant for the neutral surfactant CocoDEA afforded high surface tension values at each tested concentration. The betaine surfactant by itself afforded relatively high surface tension values. Surprisingly, the reaction product was operable to decrease the surface tension somewhat in comparison to the betaine surfactant alone.

Example 4: Substitution of CocoDEA with Ethoxylated Alcohol Neutral Surfactant. Sample C″ was prepared in the same manner as Sample C above using the procedure of Example 1A and similar reagent proportions, except substituting an ethoxylated alcohol neutral surfactant (Tomadol 1-9) for CocoDEA and conducting the reaction at 50° C. Table 10 summarizes the surface tension of the reaction product in comparison to the ethoxylated alcohol surfactant alone.

TABLE 10

| | ST at 1 gpt | ST at 2 gpt |
|---|---|---|
| Sample | (dynes/cm) | (dynes/cm) |
| Ethoxylated | 46.6 | 39.5 |
| alcohol | | |
| surfactant | | |
| Sample C″ | 47.4 | 41.9 |

The ethoxylated alcohol surfactant afforded much higher surface tension values at each tested concentration than did a like concentration of CocoDEA. The reaction product in combination with the ethoxylated alcohol surfactant afforded a similar surface tension to that of the ethoxylated alcohol surfactant alone.

Example 5: Decreased CocoDEA Concentration. Sample C′″ was prepared in the same manner as Sample C above using the procedure of Example 1A and similar reagent proportions, except the CocoDEA concentration was lowered to one-fifth the concentration used above (i.e., 1 wt. %). Table 11 summarizes the surface tension of the reaction product in comparison to the reduced-concentration CocoDEA surfactant solution alone.

TABLE 11

| | ST at 1 gpt | ST at 2 gpt |
|---|---|---|
| Sample | (dynes/cm) | (dynes/cm) |
| CocoDEA at ⅕ | 71.96 | 67.87 |
| concentration | | |
| Sample C″′ | 65.84 | 59.05 |

Lowering the CocoDEA concentration significantly increased the surface tension values. Even though the surface tension was considerably higher than when 5 wt. % CocoDEA was present, the reaction product still decreased the surface tension in comparison to CocoDEA itself.

Foaming Performance of Dextrin Reaction Products. Sample 1C (reaction product of maltodextrin and lauric acid) was processed into a soap formulation having the following composition: 61.1% wt. % deionized water, 20.9 wt. % maltodextrin/lauric acid reaction product (combined as aqueous mixture prepared as above), 7.5 wt. % cocamidopropyl betaine, 0.5 wt. % glycerin, and 10.0 wt. % SOPALTERIC CS (sodium cocoamphohydroxypropylsulfonate, Southern Chemical and Textile). A comparative soap formulation having the following composition was prepared for side-by-side evaluation of foaming performance: 20 wt. % of a 30 wt. % sodium lauryl sulfate solution in water, 5 wt. % cocoamidopropyl betaine, 0.5 wt. % glycerin, 0.8 wt. % NaCl and balance deionized water. The soap formulations contained approximately equivalent amounts of the maltodextrin/lauric acid reaction product and sodium lauryl sulfate.

Foaming performance of the experimental soap formulation in comparison to the comparative soap formulation was assayed using the Hart-DeGeorge Foam Test. In brief, the Hart-DeGeorge Foam Test utilizes a wire screen placed between a funnel and a graduated cylinder. A set volume of a foamed mixture is then introduced into the funnel, and the time required for the wire screen (850 μm mesh size) to be exposed is measured. The liquid level in the graduated cylinder is also measured at various times. Lower density foams are thus characterized by longer times required to expose the wire screen, and lower amounts of liquid collected in the graduated cylinder are indicative of a more stable foam.

To conduct Hart-DeGeorge Foam Tests with the experimental and comparative soap formulations, 1% active solutions of each soap formulation were prepared in separate 200 mL quantities of deionized water (soft water) at 25ºC. The solutions were then blended at high speed in a blender for 1 minute. At the completion of blending, the resulting foam was transferred to the funnel. The time required for the wire mesh to be exposed was measured. In addition, the liquid level in the graduated cylinder was recorded at 1, 2, 3, 4, 5 and 14 minutes. Table 12 summarizes the Hart-DeGeorge Foam Test performance of the experimental and comparative soap formulations.

TABLE 12

| | Comparative Soap | Experimental Soap |
|---|---|---|
| | Formulation | Formulation |
| Wire Time (s) | 98 | 91 |
| Liquid Volume-1 min. (mL) | 1 | 1 |
| Liquid Volume-2 min. (mL) | 1 | 1 |
| Liquid Volume-3 min. (mL) | 1 | 1 |
| Liquid Volume-4 min. (mL) | 25 | 1 |
| Liquid Volume-5 min. (mL) | 30 | 1 |
| Liquid Volume-14 min. (mL) | 125 | 105 |

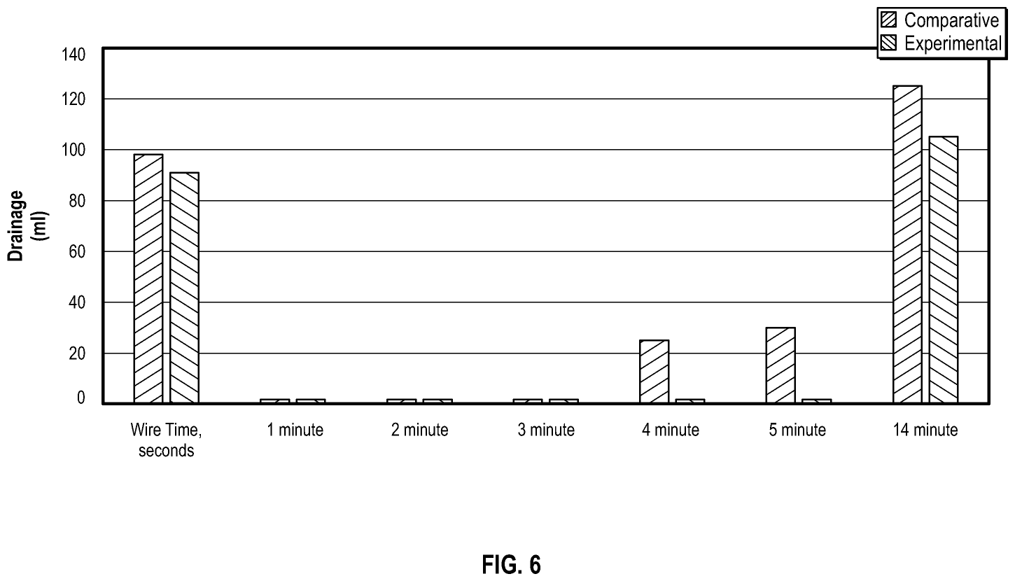

The wire time data and liquid volume data is plotted in the bar graph shown in FIG. 6. As shown, the experimental and comparative soap formulations afforded similar wire time performance at substantially equivalent surfactant concentrations, thereby indicating a similar foam density. The experimental soap formulation, in contrast, afforded a superior foam as evidenced by the lower liquid volume collected in the graduated cylinder.

Replacement of Ethoxylate Alcohol Surfactants. A reaction product was formed by reacting maltodextrin with a mixture of dodecanoic acid (C₁₂ fatty acid) and myristic acid (C₁₄ fatty acid) in the presence of CocoDEA under the general conditions specified above. The reaction product was an opaque fluid, and no settling was observed. The reaction product was formulated at a standard concentration (Sample BB), as well as at half the standard concentration and double the standard concentration (Samples AA and CC, respectively). Surface tension, intrafacial tension, and contact angle values for these fluids are specified in Table 13 below.

Surface tension, intrafacial tension, and contact angle values for three friction-reducing fluids containing ethoxylated alcohol surfactants are also shown in Table 12 (Fluids 1-3).

The ethoxylated alcohol surfactants in friction-reducing fluids 1-3 were replaced with an equivalent quantity of reaction product obtained from double-concentration Sample CC. Surface tension, intrafacial tension, and contact angle values for the modified oilfield friction reducing fluids are specified in Table 12. The modified fluids are designated Fluids 1′, 2′ and 3′, respectively.

TABLE 13

| | | Surface | Intrafacial | |
|---|---|---|---|---|
| | Concentration | Tension | Tension | Contact |
| Sample | (gpt) | (dynes/cm) | (dynes/cm) | Angle (°) |
| AA | 1 | 28.1 | 1.4 | 20.3 |
| | 2 | 28.0 | 0.6 | n/d |
| BB | 1 | 32.7 | 3.4 | 27.2 |
| | 2 | 29.8 | 1.8 | n/d |
| CC | 1 | 40.2 | 7.0 | 39.8 |
| | 2 | 32.8 | 3.8 | n/d |
| Fluid 1 | 1 | 31.66 | 2.02 | 31.3 |
| | 2 | 29.45 | 1.11 | n/d |
| Fluid 2 | 1 | 33.96 | 3.17 | 33.7 |
| | 2 | 30.60 | 2.06 | n/d |
| Fluid 3 | 1 | 30.45 | 1.85 | 30.8 |
| | 2 | 28.76 | 0.98 | n/d |
| Fluid 1′ | 1 | 28.25 | 0.36 | 29.6 |
| | 2 | 27.79 | 0.27 | n/d |
| Fluid 2′ | 1 | 30.06 | 0.74 | 33.2 |
| | 2 | 28.14 | 0.56 | n/d |
| Fluid 3′ | 1 | 28.98 | 0.41 | 28.9 |
| | 2 | 27.79 | 0.30 | n/d |

As shown in Table 13, replacement of the ethoxylated alcohol surfactant in Fluids 1-3 with a reaction product of the present disclosure afforded considerably lower surface tension and intrafacial tension values in each case. Surprisingly, the surface tension and intrafacial tension values were even lower than in the Sample CC reaction product itself. Moreover, the friction-reducing properties of Fluids 1′-3′ were not significantly changed from original Fluids 1-3 (data not shown).

Moisturizer Formulation. Dextran functionalized with lauric acid in accordance with the disclosure above was formulated as a moisturizer. A single application of the test moisturizer (50 μl) was applied to the lower leg of 32 test subjects, and the moisture content at the application site was monitored over 24 hours. Moisture content at the application site was assessed using a COSMEOMETER® CM825 (Courage+Khazaka) instrument. No moisturizer (negative control) and glycerin (humectant positive control) were evaluated similarly within the same group of test subjects. Table 14 shows the moisture content of the various test sites as a function of time.

TABLE 14

| | Mean COSMEOMETER ® Reading at Time (hr) | | | | | |
|---|---|---|---|---|---|---|
| Sample | 0 | 1 | 3 | 8 | 12 | 24 |
| Test | 27.4 | 59.5 | 56.9 | 51.7 | 45.8 | 33.5 |
| Moisturizer | | | | | | |
| Negative | 26.9 | 26.7 | 27.0 | 25.6 | 24.2 | 22.0 |
| Control | | | | | | |
| Positive | 26.4 | 144.6 | 127.5 | 116.8 | 103.0 | 92.2 |
| Control | | | | | | |

As demonstrated by the data in Table 14, a single application of the test moisturizer maintained higher moisture levels at the application site relative to the negative control for times up to 24 hours.

Unless otherwise indicated, all numbers expressing quantities and the like in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating various features are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While various systems, compositions, tools and methods are described herein in terms of “comprising” various components or steps, the systems, compositions, tools and methods can also “consist essentially of” or “consist of” the various components and steps.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Therefore, the disclosed systems, compositions, tools and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems, compositions, tools and methods illustratively disclosed herein may suitably be practiced in the absence of any element that is not specifically disclosed herein and/or any optional element disclosed herein. While systems, compositions, tools and methods are described in terms of “comprising,” “containing,” or “including” various components or steps, the systems, tools and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

What is claimed is the following:

1. A consumer or industrial product comprising:
a carrier phase;
a neutral surfactant or a reaction product thereof combined with the carrier phase; and
a reaction product of a saccharide polymer and a fatty acid or a fatty ester combined with the carrier phase, the saccharide polymer comprising a dextran, a dextrin compound, or any combination thereof, and the fatty acid comprising about 50 wt. % or above of one or more straight-chain fatty acids.

2. The consumer or industrial product of claim 1, wherein the reaction product of the saccharide polymer and the fatty acid or the fatty ester is present at a concentration effective to lower a surface tension of the neutral surfactant in an aqueous fluid.

3. The consumer or industrial product of claim 1, wherein the saccharide polymer comprises a dextrin compound and the dextrin compound comprises a maltodextrin.

4. The consumer or industrial product of claim 3, wherein the maltodextrin has a dextrose equivalent value of about 3 to about 25.

5. The consumer or industrial product of claim 1, wherein the fatty acid or a fatty acid component obtainable from the fatty ester comprises about 4 to about 30 carbon atoms.

6. The consumer or industrial product of claim 1, wherein the fatty acid or a fatty acid component obtainable from the fatty ester consists of one or more straight-chain fatty acids comprising about 4 to about 30 carbon atoms.

7. The consumer or industrial product of claim 1, wherein the fatty acid or a fatty acid component obtainable from the fatty ester comprises at least one fatty acid selected from the group consisting of butyric acid, valeric acid, caproic acid, enanthic acid, caprylic acid, pelabonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, trioscylic acid, lignoceric acid, pentacosylic acid, cerotic acid, carboceric acid, montanic acid, nonacosylic acid, melissic acid, crotonic acid, cervonic acid, linoleic acid, linolelaidic acid, linolenic acid, arachidonic acid, docosatetraenoic acid, myristoleic acid, palmitoleic acid, sappenic acid, vaccenic acid, paullinic acid, oleic acid, pinolenic acid, stearidonic acid, eleostearic acid, elaidic acid, gondoic acid, gadoleic acid, erucic acid, eicosenoic acid, eicosadiencoic acid, eicosatrienoic acid, eicosatetraenoic acid, docosadienoic acid, nervonic acid, mead acid, adrenic acid, and any combination thereof.

8. The consumer or industrial product of claim 1, wherein the neutral surfactant comprises a fatty acid alkanolamide or a reaction product thereof.

9. The composition of claim 8, wherein the fatty acid alkanolamide comprises a compound selected from the group consisting of cocamide diethanolamine, cocamide monoethanolamine, cocamide diisopropanolamine, and any combination thereof.

10. The consumer or industrial product of claim 1, wherein a molar ratio of fatty acid to saccharide polymer in the reaction product is about 0.2 or above on a basis of moles_{fatty acid}:moles_{glucose monomers}.

11. The consumer or industrial product of claim 1, wherein a molar ratio of fatty acid to saccharide polymer in the reaction product ranges from about 0.2 to about 0.8 on a basis of moles_{fatty acid}:moles_{glucose monomers}.

12. The consumer or industrial product of claim 1, wherein the reaction product of the saccharide polymer is obtained in the presence of water and a hydroxide base.

13. The consumer or industrial product of claim 1, wherein the reaction product of the saccharide polymer comprises a fatty ester saccharide polymer reaction product.

14. The consumer or industrial product of claim 1, wherein the consumer or industrial product is foamed or foamable.

15. The consumer or industrial product of claim 1, wherein the consumer or industrial product is emulsified.

16. The consumer or industrial product of claim 1, wherein the consumer or industrial product is in a stick form.

17. The consumer or industrial product of claim 1, wherein the consumer or industrial product is formulated as a cream or a gel.

18. The consumer or industrial product of claim 1, further comprising:
at least one fatty acid carboxylate.

19. The consumer or industrial product of claim 1, further comprising:
at least one zwitterionic surfactant.

20. The consumer or industrial product of claim 1, wherein the reaction product is formed from a fatty ester, and the consumer or industrial product further comprises glycerol.
